(19) United States
(12) Patent Application Publication
(10) Pub. No.: US 2018/0333724 A1
(43) Pub. Date: Nov. 22, 2018

(54) ASSAY SYSTEMS AND METHODS FOR PROCESSING SAMPLE ENTITIES

(71) Applicant: Bioelectronica Corporation, Reno, NV (US)

(72) Inventors: Jonathan F. HULL, Reno, NV (US); Martin TOMASZ, Los Angeles, CA (US); Roger CHEN, Saratoga, CA (US)

(21) Appl. No.: 15/986,416

(22) Filed: May 22, 2018

Related U.S. Application Data
(60) Provisional application No. 62/625,170, filed on Feb. 1, 2018.
(60) Provisional application No. 62/509,638, filed on May 22, 2017.

(51) Int. Cl. B01L 3/00; C12Q 1/02; G01N 27/22

(52) U.S. Cl. CPC B01L 3/502784; C12Q 1/02; G01N 27/221; B03C 2201/00; B01L 2300/0645; B01L 2300/0819; B01L 2400/0424; B01L 2200/14

(57) ABSTRACT

A system for processing sample entities includes a chamber including a surface having an array of measurement regions, wherein at least one measurement region comprises a first set of one or more electrodes and a second set of one or more electrodes, wherein the first set of electrodes is configured to measure a first characteristic of a sample entity when the sample entity is traversing the first set of electrodes, and wherein the second set of electrodes is configured to selectively retain the sample entity in the at least one measurement region based at least in part on the measured first characteristic and/or measure a second characteristic of the sample entity.

### CROSS-REFERENCE TO RELATED APPLICATIONS

This application claims priority to U.S. Patent Application Ser. No. 62/625,170, filed on Feb. 1, 2018, and to U.S. aPtent Application Ser. No. 62/509,638, filed on May 22, 2017, each of which is hereby incorporated by this reference in its entirety.

### TECHNICAL FIELD

This invention relates generally to the field of digital assays for processing sample entities.

### BACKGROUND

Assay devices are commonly used in research, diagnostic, and other applications to detect and/or measure one or more components of a sample. A digital assay is one kind of assay device that partitions a biological sample into multiple smaller containers such that each container contains a discrete number of biological entities. For example, a microfluidic digital assay may be used to analyze microfluidic droplets including single cells or other entities, such as for quantifying nucleic acids, proteins, or other biological content.

Current microfluidic systems have a number of drawbacks. For example, many droplet microfluidic systems are based on electrowetting on dielectric (EWOD) technology. In conventional EWOD devices, droplets of liquid are actuated by modifying interfacial tension between the droplet and an electrode with an electric field applied by electrodes in the device. However, one drawback of EWOD devices is that the application of the electric field damages the droplets of liquid as they are actuated, which may alter the biochemical contents of the droplets and affect analysis.

Conventional microfluidic digital assays also require that droplets be monodisperse and of the same type (e.g., exclusively DNA) during an experiment, in order to, for example, accurately correlate measurements to analyte concentration and compare such measurements across different droplets. These devices require droplets to be pre-sorted to ensure that they are of suitable uniform size, which is time-consuming and reduces efficiency in processing droplets. Additionally, these devices include a linear, single-track microfluidic channel within which droplets travel in series for processing, which further limits the efficiency for analysis of the droplets. Accordingly, there is a need for new and improved digital assay systems and methods for processing sample entities.

### SUMMARY

Generally, in some variations, an assay device for processing sample entities includes a chamber having an array of measurement regions, where at least one measurement region includes a first set of one or more electrodes and a second set of one or more electrodes. The array of measurement regions may be on a surface (e.g., a planar surface) of the chamber. The array of measurement regions may, in some variations, include a two-dimensional grid. As described in further detail below, the first set of electrodes may be configured to measure a first characteristic (e.g., relating to size and/or shape) of a sample entity when the sample entity is traversing the first set of electrodes, and the second set of electrodes may be configured to selectively retain or otherwise manipulate the sample entity in a measurement region based at least in part on the measured first characteristic. In some variations, the assay device may additionally or alternatively include one or more image sensors configured to measure one or more characteristics of the sample entity, such as through computer vision techniques. For example, at least a portion of one or more surfaces (e.g., upper surface, lower surface) of the chamber may include a substantially optically transparent material through which an image sensor may view one or more measurement regions of the chamber. In some variations, the assay device may be configured to process (e.g., measure, track, analyze, sort, etc.) polydisperse samples, and may be configured to process sample substantially in parallel for large-scale, efficient processing.

At least one of the first set of electrodes may, in some variations, be larger than a diameter of the sample entity. For example, the first set of electrodes may include at least two elongated electrodes separated by a scanning distance. As a sample entity traverses the first set of electrodes and the scanning distance, the first set of electrodes may measure a first characteristic of the sample entity. Furthermore, at least one of the second set of electrodes may, in some variations, include interdigitated electrodes. The second set of electrodes may measure a second characteristic of the sample entity. Such electrode measurements may be performed by measuring an electrical characteristic of the sample entity after delivering a measurement current to the electrodes when the sample entity is in contact with the electrodes.

In some variations, the first characteristic may be measured based at least in part on a measured double layer capacitance of the sample entity. The first characteristic may include, for example, size and/or shape of one or more sample entities in a measurement region. Furthermore, in some variations, the first and/or second set of electrodes may be configured to measure a second characteristic of the sample entity. The second characteristic may be measured based at least in part on a measured electrical impedance of the sample entity. The second characteristic may include a property of the contents of the sample entity (e.g., chemical- and/or biological-related information about the contents of the sample entity). One, two, or any suitable number of second characteristics may be measured.

The assay device may, in some variations, further include a memory device configured to store a virtual tag associated with a sample entity, wherein the virtual tag may include one or more characteristics of the sample entity. The virtual tag may be used, for example, for tracking the sample entity as is moves within the chamber.

Furthermore, generally, a system for processing at least one sample entity may include a chamber including an array of measurement regions, where each measurement region includes at least one electrode larger than a diameter of the sample entity. The array of measurement regions may, for example, include a two-dimensional grid. The at least one electrode may be configured to measure a characteristic of the sample entity when the sample entity is traversing the at least one electrode. For example, in some variations, at least one measurement region may include at least two elongated electrodes separated by a scanning distance. Additionally or alternatively, at least one measurement region may include one or more electrodes configured to retain or otherwise manipulate the sample entity with a holding force such as a dielectrophoretic force. The system may, in some variations, by configured to process sample entities that are polydisperse (e.g., entities of different volumes).

Generally, a method for processing sample entities may include receiving a plurality of sample entities in a chamber including an array of measurement regions, where at least one measurement region includes a plurality of electrodes, measuring a first characteristic of at least one sample entity with at least a portion of the electrodes as the sample entity traverses the portion of the electrodes, and retaining or otherwise manipulating with the sample entity in the at least one measurement region based at least in part on the measured first characteristic. The method may, in some variations, be used to process sample entities that are polydisperse.

In some variations, receiving the plurality of sample entities comprises deforming at least one sample entity to increase the area of contact between the sample entity and a surface of the chamber. For example, the shape of the sample entity may be altered by virtue of compression between opposite wall surfaces of the chamber, and/or with a dielectrophoretic force.

Measuring the first characteristic may be performed at least in part by delivering a measurement current from the portion of electrodes to the sample entity traversing the portion of electrodes, and analyzing an electrical characteristic of the sample entity after delivering the measurement current. In some variations, retaining the sample entity in a measurement region may include generating a dielectrophoretic force with at least a portion of the electrodes. Furthermore, at least some of the electrodes may measure a second characteristic of the sample entity, such as after the sample entity is retained on a measurement region.

In some variations, the method may further include creating and/or storing a virtual tag associated with a sample entity, where the virtual tag includes one or more characteristics of the sample entity (e.g., a first characteristic such as size or shape, a second characteristic such as impedance or other electrical characteristic, characteristics correlatable to an electrical characteristic, etc.).

Furthermore, the method may, in some variations, include sorting the plurality of sample entities. For example, a first portion of the sample entities may be selectively retained on one or more measurement regions. In some variations, sorting may involve introducing a fluidic current into the chamber to manipulate a second portion of the sample entities different from the retained first portion of the sample entities. Additionally or alternatively, sorting may involve tilting the chamber to manipulate a second portion of the sample entities different from the retained first portion of the sample entities.

Generally, another variation of a system for processing sample entities may include a chamber comprising a first surface and a second surface offset from the first surface, wherein the first and second surfaces are configured to compress a sample entity into a flattened pod. At least one of the first and second surfaces comprises an optically transparent material (e.g., glass).

The chamber may include an inlet configured to receive a plurality of sample entities, and may further include an outlet configured to release at least a portion of the received sample entities. The system may include a fluidic control system for manipulating sample entities. For example, the fluidic control system may include a fluidic pump configured to create a fluidic pressure differential between the inlet and the outlet. In some variations, the system of claim 38, wherein the fluidic pump is a vacuum pump fluidically connected to the outlet of the chamber.

In some variations, the system may include an array of measurement regions comprising at least one electrode, where the electrode may be configured to perform one or more electrode measurements. For example, in some variations, a first surface of the chamber may include a circuit board (e.g., flexible circuit board) comprising the array of measurement regions, and while a second surface comprises the optically transparent material.

Furthermore, the system may include an image sensor, such as an optical image sensor, arranged to capture at least a portion of the chamber and for enabling camera-based measurements of sample entities within the chamber. For example, in some variations, a focal plane of the image sensor is substantially coincident with one of the first surface and the second surface of the chamber. As another example, a focal plane of the image sensor is located between the first surface and the second surface of the chamber. In some variations, the system may further include an illumination source, where the illumination source and the image sensor are arranged on opposing surfaces of the chamber. The illumination source may, for example, provide backlighting against sample entities within the chamber and improve quality of camera-based measurements.

### BRIEF DESCRIPTION OF THE DRAWINGS

FIGS. 1A and 1B are schematic illustrations of two exemplary variations of an assay device.

FIGS. 2A and 2B are schematic illustrations of an empty assay device and an assay device being filled with a plurality of sample entities.

FIG. 3 is a schematic illustration of a fluidic system for manipulating sample entities in an assay device.

FIGS. 4A and 4B are schematic illustrations of an exemplary variation of an assay device including cameras. FIGS. 4C and 4D are perspective and side view schematic illustrations of another exemplary variation of an assay device including cameras.

FIG. 5A is a schematic illustration of an exemplary variation of an array of measurement regions. FIG. 5B is a detailed view of electrodes in a measurement region of FIG. 5A.

FIG. 6A is a circuit schematic of electrode measurement of a pod.

FIGS. 6B-6D are schematic illustrations of a pod progressively traversing an exemplary variation of slit scanning electrodes, where the slit scanning electrode perform a scanning measurement of the pod.

FIGS. 7A-7E illustrate how different pod sizes and/or shapes result in different measurement waveforms as the pod(s) traverse slit scanning electrodes. FIG. 7A illustrates a small pod, corresponding to measurement waveform (a) depicted in FIG. 7E. FIG. 7B illustrates a large pod, corresponding to measurement waveform (b) depicted in FIG. 7E. FIG. 7C illustrates two pods traversing the slit scanning electrodes in parallel, corresponding to measurement waveform (c) depicted in FIG. 7E. FIG. 7D illustrates two pods traversing the slit scanning electrodes in series, corresponding to measurement waveform (d) depicted in FIG. 7E. FIG. 7E depicts an illustrative set of measurement waveforms for different pod sizes and/or shapes.

FIG. 8A is a schematic illustration of an exemplary variation of interdigitated electrodes. FIG. 8B is a schematic illustration of pods being deformed via a PDEP force. FIGS. 8C and 8D illustrate a measurement current waveform and a voltage waveform, respectively, that may be used to determine impedance of a pod. FIGS. 8E and 8F are exemplary measured voltage waveforms illustrating different impedance of two different samples.

FIG. 9 is a schematic illustration of an exemplary variation of a control system for controlling an array of measurement regions.

FIG. 10 is a schematic illustration of another exemplary variation of an array of measurement regions.

FIG. 11 is a schematic illustration of another exemplary variation of an array of measurement regions.

FIG. 12 is a flowchart of an exemplary variation of a method for processing sample entities.

FIGS. 13A and 13B are schematic illustrations of unactivated and activated pods, respectively, in the context of enabling differential pod measurements.

FIGS. 14A-14C are schematic illustrations of one variation of sorting pods.

FIGS. 15A and 15B are schematic illustrations of another variation of sorting pods.

FIGS. 16A and 16B are schematic illustrations of another variation of sorting pods using gravity.

FIG. 17 is a schematic illustration of an exemplary handheld variation of an assay device.

FIG. 18 is a schematic illustration of another exemplary variation of an assay device.

FIG. 19 is an exemplary illustration of computer vision techniques to measure pod size.

FIG. 20A is a schematic illustration of a pod without agglutination. FIG. 20B is an illustrative histogram of pixel grayscale intensity values of an image of the pod depicted in FIG. 20A. FIG. 20C is an illustrative histogram of size of entities in the pod shown in FIG. 20A.

FIG. 21A is a schematic illustration of a pod with agglutination. 21B is an illustrative histogram of pixel grayscale intensity values of an image of the pod depicted in FIG. 21A. FIG. 21C is an illustrative histogram of size of entities in the pod shown in FIG. 21A.

FIG. 22A is an exemplary optical image of pods without agglutination. FIG. 22B is a histogram of pixel grayscale intensity of a pod depicted in FIG. 22A.

FIG. 23A is an exemplary optical image of pods with agglutination. FIG. 23B is a histogram of pixel grayscale intensity of a pod depicted in FIG. 23A.

### DETAILED DESCRIPTION

Non-limiting examples of various aspects and variations of the invention are described herein and illustrated in the accompanying drawings.

Generally, described herein are exemplary variations of assay systems and methods for processing sample entities. For example, such systems and methods may process a large number of sample entities substantially in parallel, such as to enable rapid experimental analysis of the sample entities. Furthermore, the systems and methods described herein may be used to process polydisperse sample entities of non-uniform size. Generally, the systems and methods described herein may facilitate measurements of diagnostic- and/or research-related events or sample characteristics, such as agglutination, colloidal stability, cell growth, cell surface profiling, cell size profiling, and/or the profiling of concentration of proteins, antibiotics, nucleotides, other analytes, and the like. Applications may include diagnostics, drug research, environmental research, and the like.

As described in further detail below, the systems and methods may, for example, process sample entities, or partitioned samples. Such sample entities, a type of which is also referred to herein as “pods,” may be any suitable experimental vesicle. A pod may include in its body any suitable experimentally useful content, such as cells, DNA, RNA, nucleotides, proteins, enzymes, and/or any suitable chemical and/or biological content for analysis. In other examples, a pod may include reagents that are used to confer signals to electrodes in the assay device (and/or to cameras) such that the pods may be processed by software to yield meaningful chemical and/or biological information. Suitable reagents or agglutinates may include, for example, beads coated with gold, latex, cellulose, agarose, and/or other materials bound to biologically active proteins or scaffolds (e.g., materials suitable for ELISA kits and agglutination assays such as cell surface binding and cell agglutination assays). The degree of agglutination resulting from self-aggregation of such reagents or agglutinates (which may be monodisperse or polydisperse) in the assay system described herein may, for example, enable inference of protein and/or analyte concentrations.

In some variations, each pod may be considered a separate experiment, such that processing of multiple pods enables the fast and efficient performance of multiple experiments in parallel. Processing pods may involve, without limitation, analyzing one or more characteristics of pods, tracking location and/or predicting trajectory of pods within the chamber, and/or manipulating pods for sorting.

In some variations, a pod may include an aqueous phase that is stabilized and is transportable in a surrounding medium such as a liquid or other fluid (e.g., surfactant or lipid). In some variations, a pod being processed by the assay device may be distinct from a droplet at least in part because a pod is not spherical. For example, a processed pod might not be spherically symmetrical. The processed pod may be smaller in one dimension (e.g., in a dimension measured generally orthogonal to an electrode surface as described below) than in another dimension. For example, the processed pod may be generally flattened on at least one side, similar to a generally hemi-spherical shape, or may be generally flattened on at least two opposing sides, similar to a disk-like or “pancake” shape. As described in further detail below, a pod that is flattened on at least one side may have increased surface area of contact with measurement electrodes in the assay device, such that electrode measurements may have reduced noise and generally improved signal quality. Additionally, as described in further detail below, a pod that is flattened on at least one side may be volumetrically restricted so as to concentrate the pod contents into a shape approximating a two-dimensional focal plane of a camera, thereby improving visibility of the pod contents by the camera. Furthermore, a pod may be distinct from a droplet at least in part because multiple pods being processed simultaneously by the assay device may be polydisperse, in contrast to droplets which are conventionally thought of as being the same size (e.g., having monodisperse characteristics).

For example, a pod may be pressed into a flattened form (e.g., by mechanical compression between two plates or other suitable mechanism), by increasing surfactant concentration, and/or with a positive dielectrophoretic (PDEP) force as described in further detail below.

The surrounding medium for the pods may, for example, include a non-aqueous continuous phase. In some variations, the surrounding medium may be fluorous. For example, the medium may include a fluorinated oil or other liquid (e.g., HFE 7500 available as Novec™ manufactured by 3M™ or FC-40, available as Fluorinert™ manufactured by 3M). As another example, the medium may include hydrocarbon oil. The medium may, in yet other variations, additionally or alternatively include PEG and fluoridated derivatives (e.g., derivatives of Krytox™ fluorinated oils manufactured by The Chemours Company, which may be polymerized or co-polymerized with PEG or other suitable glycol ethers), and may include lipids or other phosphoric, carboxylated or amino-terminated chains.

In some variations, a pod may have an overall density that is lower than the density of the surrounding medium, such that aqueous pods within the medium are more buoyant and tend to rise within the surrounding medium. For example, the surrounding medium may include a fluid denser than water, such as HFE-7500 and/or FC-40, which may be mixed with co-block polyethylene glycol/Krytox™ polymer. In other variations, a pod may have an overall density that is higher than the density of the surrounding medium such that aqueous pods within the medium are less buoyant tend to sink within the surrounding medium. For example, the surrounding medium may include a fluid less dense than water, such as hexadecane and a phospholipid bilayer. In yet other variations, a pod and its surrounding medium may have substantially similar or equal densities. It should be understood that various combinations of relative densities of pods and the surrounding medium may provide varying levels of buoyancy of the pods within the surrounding medium (e.g., a set of pods within a particular medium may include some pods that tend to rise and some pods that tend to sink). For example, relative buoyancy of the pods may be beneficial in some applications to leverage gravity in sorting of pods. However, the pod may be surrounded by any suitable medium.

One or more pods may be introduced in combination with a suitable surrounding medium as an emulsion into an assay device and processed as described herein. In some variations, mixing to create pods may occur outside of the assay device (e.g. adjacent an external side of an inlet of the device prior to introduction into the device), while in other variations such mixing may additionally or alternatively occur inside the assay device. For example, pods may be generated by agitating two solutions including a biological reagent and a fluorinated liquid. Furthermore, larger pods may be transformed into smaller pods (e.g., by interaction with support posts in the assay device as described below, or interaction with any other suitable device feature) to control or adjust polydispersity among the pods.

The assay devices and methods may be used to process polydisperse sample entities. For example, various aspects of the devices and methods described herein may enable substantially simultaneous processing of pods of different sizes, in contrast to conventional systems which require samples to be monodisperse. In some variations, the assay devices and methods described herein may simultaneously process sample entities having at least 5%, at least 10%, at least 25%, or at least 50% variance in size (e.g., pod diameter, pod circumference, pod surface area, pod volume, etc.). The ability to handle polydisperse samples may, for example, provide sample analysis that is simpler and more efficient (e.g., by not requiring the sample entities to be sorted by size in a separate, time-consuming process before introducing them into an assay device).

Exemplary applications of the assay devices and methods described herein include processing pods to measure analyte concentration, measure cell division, measure morphology, size, and/or number of cells or particles within a pod or other sample entity, measure relative sizes of cells and the pods within they are contained (e.g., ratio between circumference of a pod and the circumference of a cell within the pod), and the like. For example, the devices and methods may be used for pathology, oncology, determining white or red blood cell counts, etc. Furthermore, the assay devices and methods described herein may be used to perform any of a wide variety of agglutination tests.

### Systems for Processing Sample Entities

Generally, in some variations, an assay device for processing sample entities includes a chamber having an array of one or more measurement regions. In some variations, at least one measurement region may include a first set of one or more electrodes and a second set of one or more electrodes. The electrodes in each measurement region may be independently operable, such that each measurement region may provide data independently of each other. The array of measurement regions may be on a surface (e.g., a planar surface) of the chamber. As described in further detail below, the first set of electrodes may be configured to measure a first characteristic (e.g., relating to size and/or shape) of a sample entity when the sample entity is traversing the first set of electrodes, and the second set of electrodes may be configured to selectively retain or otherwise manipulate the sample entity in a measurement region based at least in part on the measured first characteristics. Furthermore, in some variations, the first set of electrodes and/or second set of electrodes may be configured to measure a second characteristic of the sample entity (e.g., relating to chemical and/or biological information of the sample entity). One or more cameras may, in some variations, additionally or alternatively be used to measure one or more characteristics of sample entities, such as through suitable computer vision techniques.

### Chamber

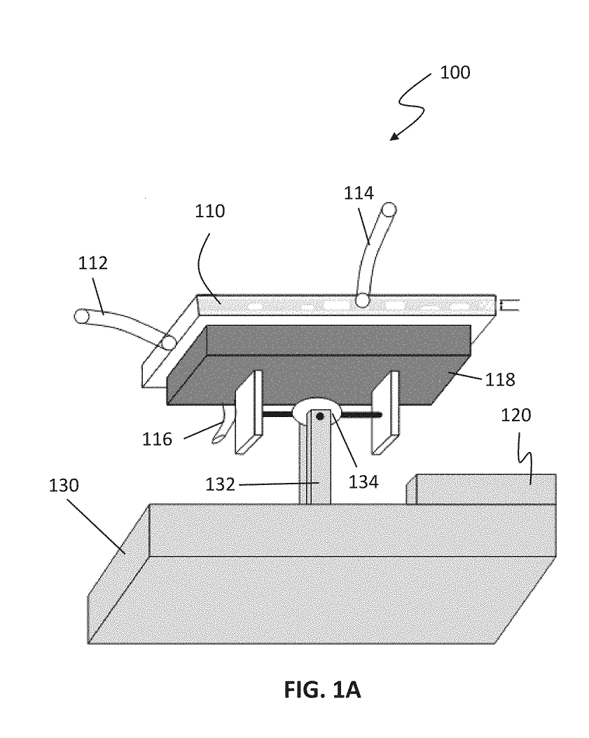

As shown in FIG. 1A, an assay device 100 may include at least one chamber 110 including at least one inlet 112 and at least one outlet (e.g., outlet 114, outlet 116, etc.). The chamber 110 may be configured to receive one or more pods for processing. For example, an emulsion of pods and their surrounding medium may be passed into the chamber 110 via the inlet 112 (e.g., with a suitable pump) to circulate within the chamber 110. As the emulsion of pods and their surrounding medium circulates within the chamber 110, the pods may traverse and make contact with any of a plurality of measurement regions that are disposed on a surface of the chamber 110 (e.g., a surface that bounds at least one side of the enclosed volume). The measurement regions may, for example perform electrode measurements of the pods. Additionally or alternatively, cameras may be disposed proximate the chamber to enable camera-based (e.g., optical) measurements of the pods. Once analyzed or otherwise processed within the chamber 110 (e.g., based at least in part on electrode and/or camera-based measurements as further described below), the pods may be sorted by being passed out of the chamber 110 via the one or more outlets 114 and 116.

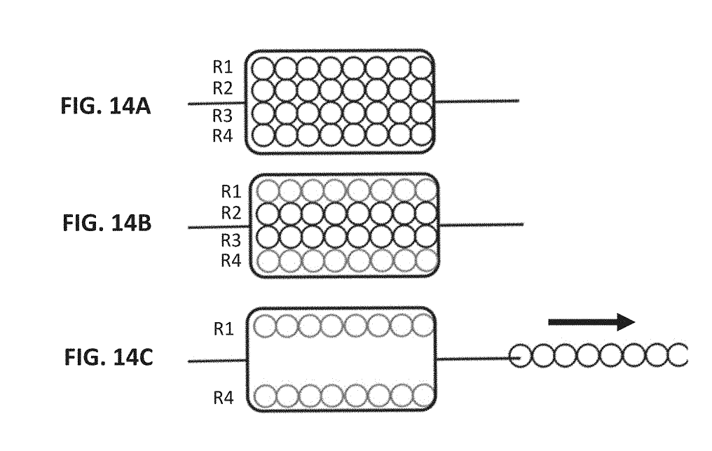

Although the assay device 100 is depicted as including only one chamber, it should be understood that the assay device may be modular and include multiple chambers. In some variations, multiple chambers (or multiple assay devices 100) may operate in series such that one reaction or event may be measured in a first chamber and a second reaction or event may be measured in a second chamber. For example, after a set of pods are processed in a first run through a first chamber least some of the pods may be treated (e.g., cell contents lysed) to prepare the treated pods for processing in a second chamber. In this manner, pods may be processed serially in different matters. Additionally or alternatively, in some variations, multiple chambers may operate in parallel. For example, the device may include two, three, four, or more chambers operating as separate panels that may be used in parallel to increase the device's processing capacity, and/or may be used in series to process pods in stages (e.g., different experiments). For example, as shown in FIG. 17, a handheld assay device 1710 may include multiple panels 1720a-1720e that are stacked in layers in a single device. Each panel may have its own respective chamber with measurement regions and electrodes, etc., similar to that described herein. For example, in some variations, the device 1710 may be used in diagnostic applications to provide specific diagnoses (e.g., strep throat, influenza, prostate cancer, etc.), such as in point-of-care or point-of-need testing. In some variations, the handheld assay device 1710 may be disposable.

In some variations, the chamber 110 may define an enclosed volume that is expansive such that pods entering the chamber may travel freely in at least two dimensions within the chamber (e.g., substantially freely in both X- and Y-directions, in contrast to a microfluidic channel providing for substantially unidirectional flow of samples). In some variations, the chamber does not constrain the sample entities to a channel, such that the assay device may process a wide range of pod sizes not limited by the width of a microfluidic channel. The enclosed volume of the chamber 110 may, for example, be a generally prismatic volume formed by an upper surface, a lower surface, and one or more sidewall surfaces adjoining the upper and lower surfaces. For example, the enclosed volume shown in FIG. 1A is generally a rectangular prism, while in other variations, the enclosed volume of the chamber 110 may be cylindrical or any suitable shape. The upper and lower surfaces (or at least portions thereof that receive sample entities across an array of measurement regions, as described below) may, in some variations, be parallel such that the height or depth of the chamber is substantially uniform.

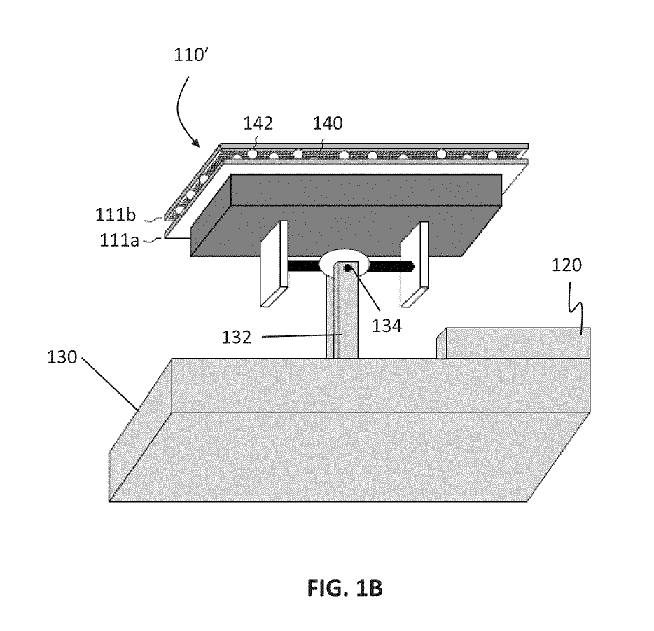

The filling of a chamber is generally shown in the illustrative schematic of FIGS. 2A and 2B. As shown in FIG. 2A, similar to FIG. 1A, a chamber 210 may include at least one inlet 212 and at least one outlet 214. Pods 202 may enter the chamber 210 via the inlet 212 and at least partially fill an interior volume of the chamber, as shown in FIG. 2B. It should be understood that the relative sizes of the chamber 210 and pods are not to scale, and that pods may not necessarily be monodisperse and may not pack the chamber 210 in the uniform manner shown in FIG. 2B. Additionally, although a single inlet 212 is shown in FIG. 1A, in other variations, the device may include multiple inlets (e.g., on one side of the chamber, distributed around the perimeter of the chamber, etc.). Once analyzed, the pods may be passed out of the chamber 210 via the outlet 214.

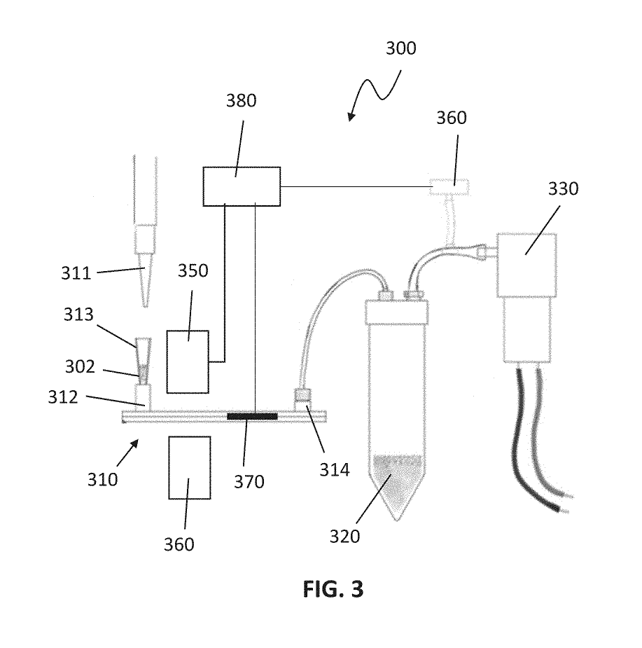

In some variations, height or depth of the chamber may contribute to formation of pods. For example, as shown in FIGS. 4C and 4D, a plurality of pods may be formed by compressing droplets between a first surface 422 and a second surface 424 (e.g., upper and lower surfaces) of the chamber 410, where the first and second surfaces are separated and offset by a gap spacing that is less than the diameter of the original droplets. In some variations, the first and second surface are offset by a gap spacing that is less than about 50 μm, less than about 25 μm, less than about 10 μm, or about 5 μm. Advantageously, the compressed shape increases the pods' surface area of contact with the measurement electrodes in the chamber, thereby improving the quality of electrode measurements, as further described below. Additionally, the compressed shape may restrict the pod contents to approximately a two-dimensional plane. As shown in FIG. 4D, this two-dimensional plane may be substantially coincident with the focal plane 430 of one or more cameras (e.g., cameras 412A and 412B), thereby improving detection of the pod contents (e.g., analytes) by the camera and improving the quality of camera-based measurements. Additionally or alternatively, a droplet may be transformed into a pod with surfactants, or through any other suitable mechanism. A pod may be formed inside or outside of the chamber.

In some variations, the chamber 110 may be tiltable or pivotable. For example, as shown in FIG. 1A, the chamber 110 may be generally supported by a base 130 which may be configured to rest on a stable, grounded surface (e.g., benchtop or desktop). Base 130 may include a pillar support 132 which is pivotably coupled to a chamber base 118 via joint 134. Alternatively, the pillar support 132 may be pivotably coupled directly to a suitable surface of the chamber 110. The joint 134 may include a pin joint as shown in FIG. 1A providing rotation about a single axis. In other variations, the joint 134 may include a multi-directional joint (e.g., spherical joint, ball-and-socket joint) or a combination of joints amounting to rotation about multiple axes. Additionally or alternatively, motion of the chamber 110 relative to the base 130 may be provided by movement of the pillar support 132 relative to the base 130. However, in other variations, the chamber 110 may be movable relative to the base 130 in any suitable manner. One or more actuators (e.g., stepper motors or servomotors), which may be combined with suitable gear train or other transmission, may be coupled to the joint 134 to electromechanically actuate movement of the chamber 110. A suitable electronic control system, shown schematically as controller 120 in FIG. 1A, may be used to control the movement of the chamber 110. Generally, the controller 120 may further be configured to execute the instructions that are stored in a memory device such that, when it executes the instructions, the controller 120 performs aspects of the methods described herein.

Another variation of a chamber 110′ is shown in FIG. 1B. The chamber 110′ may include a volume formed at least in part by a lower substrate 111a and an upper substrate 111b spaced apart from the lower substrate 111a. The lower substrate 111a and/or the upper substrate 111b may include an array of measurement regions 140 for receiving one or more pods. The lower substrate 111a and the upper substrate 111b may be separated by a gap maintained by one or more spacers 142 (e.g., glass beads) that are placed between the substrates. In some variations, the spacers 142 may control the size of the gap between the substrates (e.g., and/or to maintain a parallel relationship between the lower substrate 111a and the upper substrate 111b, to help ensure laminar flow of the pod emulsion in the chamber 110′, etc.). In some variations, the gap between the substrates may be between about 10 μm and about 1000 μm. In some variations, the gap may be adjustable to better accommodate different kinds or size ranges of pods, such as by swapping spacers 142 of different lengths or sizes. The chamber 110′ may include other suitable structures or other mechanisms for maintaining the size of the gap, such as ridges extending from the substrates, or sidewalls connecting the lower and upper substrates.

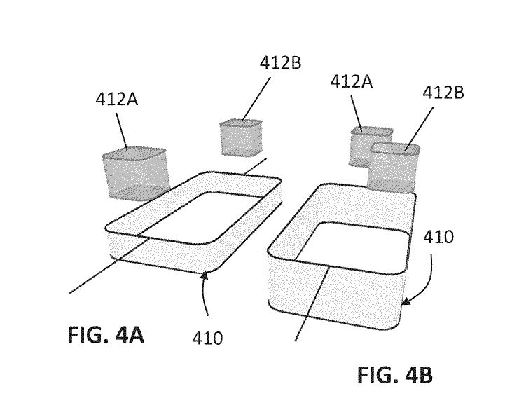

In another variation as shown in FIG. 5A, spacers similar to spacers 142 described above may include one or more support posts (cross-sections of which are shown as elements 530) acting to maintain a gap distance between an upper substrate and a lower substrate. The support posts 530 may be integrally formed with and/or coupled to one or more of the substrates (e.g., coupled to one or more of the substrates with epoxy, mechanically engaged with one or more of the substrates with interlocking or complementary shaped features, etc.). In some variations, the support posts may be arranged in a regular array as shown in FIG. 5A, but may alternatively be arranged in any suitable manner (e.g., radially symmetrically distributed, random, etc.) other than what is shown in FIG. 5A. Furthermore, the support posts may be rearrangeable for different applications (e.g., a first arrangement for a first application, a second arrangement different from the first arrangement and for a second application). In some variations, one or more support posts may interact with pods in the chamber, such as by dividing or separating pods (e.g., to control or alter the degree of dispersity among the pods).

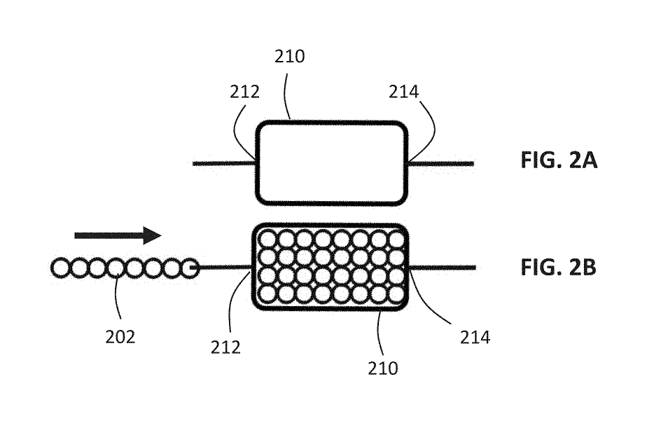

As shown in the schematic of FIG. 3, in some variations, a chamber (e.g., chamber 310) may be coupled to a fluidic control system 300 that operates to manipulate pods or other sample entities with a fluidic pressure differential. For example, the fluidic pressure differential may induce one or more pods to enter the chamber through an inlet of the chamber, induce one or more pods to traverse at least one measurement region in the chamber, and/or induce one or more pods to exit the chamber through an outlet of the chamber. Generally, the fluidic control system 300 may include a vacuum pump 330 or other pressure source fluidically coupled to the chamber 310 and configured to provide a pressure differential for manipulating pods. For example, the vacuum pump 330 may draw an emulsion 302 (including sample entities) from a reservoir 313 (e.g., tank or other container) into the chamber 310 through at least one inlet 312. The reservoir 313 holding the emulsion 302 may be coupled to the inlet 312 via threads or in any suitable manner, or may be integrally formed with the inlet of the chamber. In some variations, the emulsion 302 may be deposited or otherwise collected in the reservoir 313 with a pipette 311 (e.g., manually controlled or automatically controlled such as with a robotic system) or in any suitable manner.

The vacuum pump 330 may additionally or alternatively be configured to draw at least a portion of emulsion 302 from the chamber 310 through at least one outlet 314. As another example, the vacuum pump 330 may be used to sort or otherwise manipulate pods within the chamber 310, as further described below.

In some variations, a waste container 320 may be coupled in-line between the chamber outlet 314 and the vacuum pump 330 for receiving and holding emulsion that has exited the chamber 310. One or more valves (e.g., at the inlet 312, within the chamber 310, at the outlet 314, etc.) may enable further fluidic control within the assay device and overall system. Furthermore, one or more pressure sensors 360 (or flow sensors, or any suitable sensor) may be disposed in the fluidic system (e.g., between the waste container 320 and the vacuum pump 330, as shown in FIG. 3) to monitor pressure or other status of the fluidic system. For example, a controller 380 may implement any suitable control system to operate the vacuum pump 330 based at least in part on sensor input from pressure sensor 360, to maintain a desired rate of flow into the chamber 310. Although FIG. 3 depicts a vacuum pump 330, it should be understood that in some variations, a positive pressure pump may additionally and/or alternatively be fluidically connected on the inlet side of the chamber 310 to further facilitate a pressure differential for filling and/or otherwise manipulating the emulsion 302. Furthermore, the system may include any suitable number of pumps for any suitable number of inlets and outlets, similar to that further described below with respect to FIG. 5A.

### Measurement Regions

As shown in FIG. 5A, a chamber may include a surface 500 that includes an array of measurement regions 520. The surface may, for example, be generally planar and adjacent to an inlet 512 for receiving an emulsion of pods and surrounding medium. The surface with measurement regions 520 may be an upper surface of the chamber or a lower surface of the chamber. In some variations, both the upper and lower surfaces of the chamber (and/or any other suitable surfaces) may include measurement regions. In some variations, the array of measurement regions 520 may include electrodes that are configured to measure characteristics of pods that are in contact with the electrodes. The respective sets of electrodes in the measurement regions 520 may be individually operated, such that each measurement region 520 may provide an independent measurement of any pod in contact with it.

In some variations, the measurement regions 520 may be arranged in a rectangular array of N columns and M rows, thereby providing an array with N×M measurement regions 420. However, the measurement regions 520 may be arranged in any suitable regular or irregular manner. For example, the measurement regions 520 may alternatively be arranged in a radial array (e.g., in a plurality of rings).

At least some of the measurement regions 520 may be spaced apart from each other. For example, as shown in FIG. 5A, at least some of the N columns may be spaced apart from one another to provide spacing between measurement regions 520 in adjacent columns. In some variations, the spacing between columns may range be between about 2000 μm and 3000 μm, or about 2500 μm. The spacing between columns may be uniform or non-uniform. Such spacing may, in some variations, accommodate pods to pass between adjacent columns and exit the chamber through an outlet opening 513A, 513B, or 513C, etc. that feeds into an outlet 514, or an outlet opening 515A, 515B, or 515C, etc. that feeds into an outlet 516. For example, a pod traversing a measurement region in column 1 may exit the chamber through outlet opening 513A and toward outlet 514, or may exit the chamber through outlet opening 515A and toward outlet 516. Pods may be manipulated to exit through selected chamber outlets in a sorting process described in further detail below.

In some variations, as shown in FIG. 5B, a measurement region 520 may include a first electrode set 522 of one or more electrodes, and a second electrode set 524 of one or more electrodes. For example, the first electrode set 522 may be configured to perform a “slit scanning” measurement to measure at least a first characteristic (e.g., size, shape) of one or more pods while the one or more pods are in movement traversing the first electrode set. As another example, the second electrode set 524 may be configured to selectively retain or otherwise manipulate at least one pod in the measurement region by generating a force on the pod (e.g., based on the measured first characteristic of the pod) and/or measure a second characteristic of the pod, such as biologically-relevant parameters of the pod contents (measured as impedance of the pod's content). However, in some variations, a measurement region may include only the first electrode set 522, or only the second electrode set 524. For example, the first electrode set 522 (e.g., a slit scanning electrodes) may be configured to measure first and second characteristics of a pod, and/or apply a holding force to retain the pod in the measurement region. As another example, the second electrode set 524 (e.g., interdigitated electrodes) may be configured to measure first and second characteristics of a pod and/or apply a holding force to retain the pod in the measurement region.

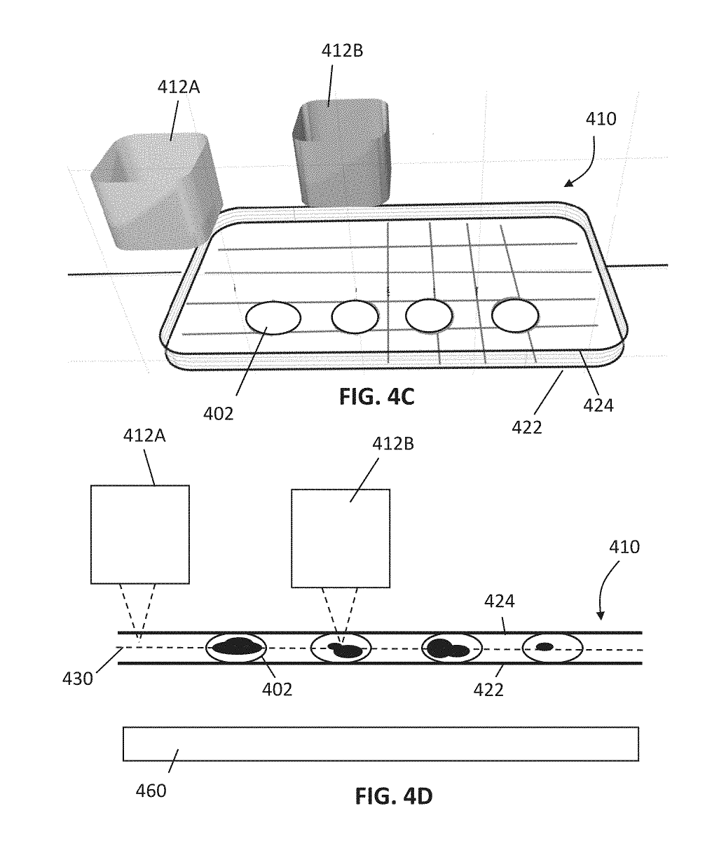

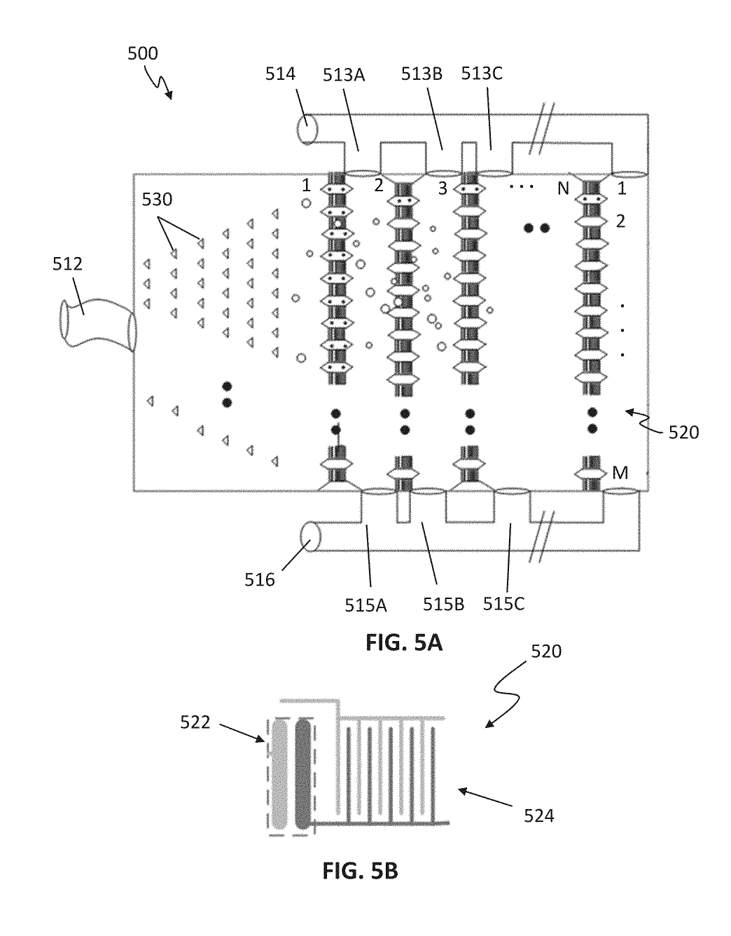

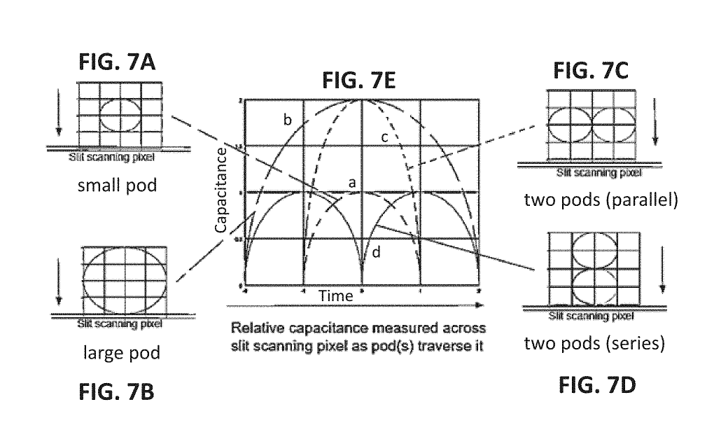

Advantageously, as shown in FIGS. 6A-6C and FIG. 8, electrodes in the measurement regions may be generally larger than the pods they receive. For example, electrodes 610 and 620 in FIGS. 6A-6C are larger than the measured pod P, and similarly, electrodes 810 and 820 in FIG. 8 are larger than the measured pod P. Because the measuring electrodes are larger than the pods they receive, the pods need not be in a narrowly prescribed region in order to be measured. Additionally, the large electrodes are more agnostic to pod size in that they are able to function and perform measurements on pods of a wider range of sizes (e.g., pods having any diameter up to the surface area of the electrode). For example, at the same time, one measurement region may be able to process a small pod, while another measurement region in the same assay device may be able to process a large pod. Accordingly, the relative sizing of the electrodes and pods help enable the assay device to process polydisperse sample entities in parallel.

Generally, electrode sets in the measurement regions 420 may perform measurements based on the circuit model illustrated in the circuit schematic of FIG. 6A. An electrode set (e.g., slit scanning electrodes, interdigitated electrodes) may include an active electrode 610 and a ground electrode 620. A complete circuit is formed when a pod P is in contact with both the active electrode 610 and the ground electrode 620, thereby enabling electronic measurement of one or more characteristics of the pod P. The interface between each electrode and the fluid content of the pod P exhibits the electrical phenomenon of double layer capacitance (represented in the schematic by capacitors C1 and C2) as part of the circuit, while the pod P itself exhibits signature impedance and other electrical characteristics (represented in the schematic by resistor R) corresponding to the nature of the pod's content. The impedance is coupled in series with the double layer capacitance. Accordingly, the active electrode 610 may apply a measurement current that travels through the pod and to the ground electrode 620, and the resulting voltages in the circuit may be measured and analyzed to determine particular characteristics of the pod. Such a measurement circuit may, for example, be suitable alternating current signal.

The flattened or compressed shape of pods may contribute to higher quality electrode measurements, in view of the schematic of FIG. 6A. For example, the flattened pod shape has an increased surface area of contact with the electrodes, which increases the magnitude of double-layer capacitance (C1, C2). Larger double-layer capacitances tends to approximate a short circuit at the frequencies typically used for measurement, reducing the variability and signal degradation of the double-layer from the measurement circuit, thereby allowing for more direct measurement of the impedance of the aqueous liquid inside the pod. In other words, the flattened pod shape may, in some variations, tend to advantageously improve the quality of measurements.

One or more electrodes of the measurement regions may be constructed in any suitable manner. For example, the measurement regions may be formed upon a measurement surface on a flexible circuit board (e.g., in a “flex circuit”). In variations in which the measurement regions may be formed in a flex circuit, the flex circuit may be supported or backed by a rigid or semi-rigid material (e.g., plastic). Additionally or alternatively, the measurement regions may be formed upon a measurement surface comprising a rigid or semi-rigid material, such as a printed circuit board including a rigid or semi-rigid substrate. Measurement regions and associated circuity (e.g., electrodes, conductive traces, switches, etc.) may be printed, soldered, and/or otherwise formed on the measurement surface.

Different exemplary types of electrodes in a measurement region 420 and their operation are described below.

### Slit Scanning Electrodes

In some variations, at least one measurement region may include a pair of slit scanning electrodes, which may be configured to measure a characteristic of at least one pod as it traverses (is in motion passing over) the slit scanning electrodes. The pair of slit scanning electrodes in one measurement region may provide a separate measurement independent of other electrodes in other measurement regions. Accordingly, in some variations, the pair of slit scanning electrodes may be referred to as a slit scanning “pixel” that provides a respective pod measurement value for processing.

As shown generally in the schematics of FIGS. 6B-6D, one variation of the slit scanning electrodes may include an active electrode 610 and a ground electrode 620. The electrodes 610 and 620 may be separated by a gap or scanning distance that may be bridged by the presence of at least one pod P in contact with both electrodes. The electrodes 610 and 620 may be generally elongated, or at least larger than the pod P to be measured. As shown in the variation of FIGS. 6B-6D, the slit scanning electrodes may be linear and generally parallel to each other such that the scanning distance along the electrodes' lengths is generally constant. However, the slit scanning electrodes may have other suitable shapes. In an exemplary variation, the slit scanning electrodes may be about 500 μm long, and the gap between the slit scanning electrodes may be about 20 μm.

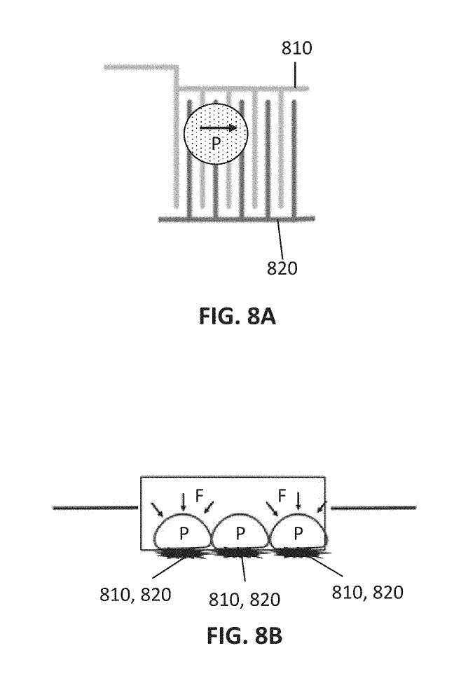

As shown in FIG. 9, a controller 910 may govern operation of the slit scanning electrodes to measure one or more pod characteristics. Generally, the controller 910 may control delivery of a measurement current to the electrodes, such as by controlling one or more switches connected to a measurement current source 940 (or alternatively intermittently driving the measurement current across a fixed connection). For example, switches 932 and 936 connect the active electrode of respective slit scanning electrode pairs to the measurement current source 940. With reference to row 2 shown in FIG. 9, a measurement current may be periodically applied across the electrodes by toggling switch 936, including while a pod traverses across the slit scanning electrodes connected to the switch 936. In some variations, for example, the current may be a DC current generally on the order of about 1 μA, but may be any suitable kind of current for applying to the electrodes. As the pod moves across the slit scanning electrodes, a corresponding voltage (or other suitable signal) may be measured and subsequently analyzed by a waveform processor 920. A similar arrangement may be repeated respectively for all measurement regions (e.g., row 1 through row M).

In some variations, the slit scanning electrodes may be configured to measure size and/or shape of at least one pod traversing the electrodes. As shown in FIGS. 6A-6C, as pod P traverses the electrodes 610 and 620, different areas and portions of the pod P overlap with each of the electrodes. As a result, capacitance rises and falls as the pod traverses across the electrodes, and a measured voltage waveform may generally track the rise and fall of the capacitance. The waveform processor 920 may analyze and interpret the nature of the waveform to determine size and/or shape of the pod. For example, shape (e.g., slope, magnitude, overall contours, etc.) of the waveform may be correlated to size of the pod.

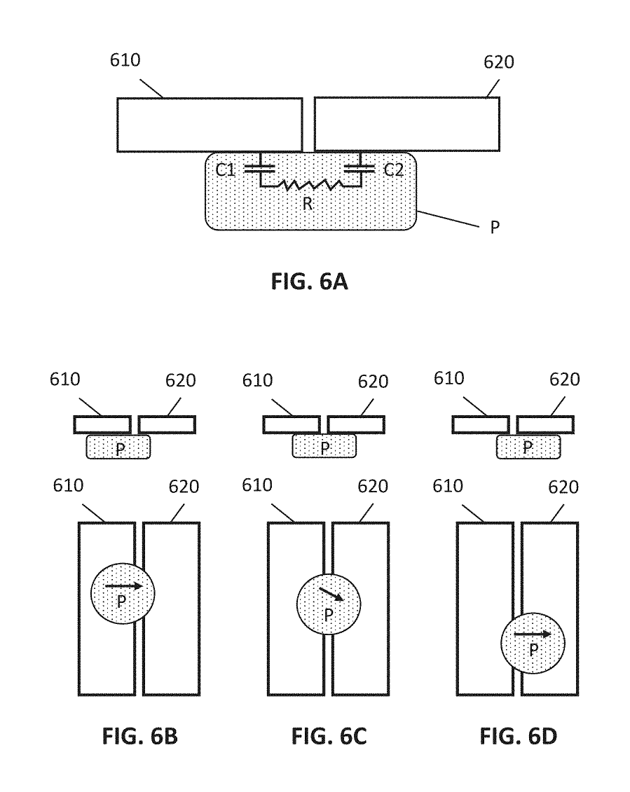

For example, comparing curves (a) and (b) in FIG. 7E, curve (a) has a briefer period of rise and fall, and a smaller maximum magnitude than curve (b). Accordingly, curve (a) corresponds to a small pod (FIG. 7A) that takes less time to fully traverse the slit scanning electrodes and also results in less capacitance due to having smaller pod volume. Curve (b) corresponds to a large pod (FIG. 7B) that takes more time to fully traverse the slit scanning electrodes and also results in more capacitance due to having more pod volume. In some variations, pod size may be determined with a lookup table correlating the measured waveform (e.g., voltage) with pod size. Additionally or alternatively, a parametric model or other suitable kind of correlating may be used to determine pod size from the measured waveform. In yet other variations, pod size may be determined with an electrode measurement model that correlates the measured waveform with pod size. Such an electrode measurement model may be trained, for example, using suitable machine learning algorithms applied to training data derived from computer vision techniques, as described in further detail below.

Additionally or alternatively, in some variations, the slit scanning electrodes may be configured to determine the shape of the one or more pods, and/or whether multiple pods are traversing the electrodes. For example, comparing curves (c) and (d) in FIG. 7E, curve (c) has a briefer period of rise and fall, and a larger maximum magnitude than curve (d). Accordingly, curve (c) corresponds to two pods traversing the slit scanning electrodes in parallel, where two pods in parallel take the same period of time to fully traverse the slit scanning electrodes as a single pod, and also results in more capacitance than a single pod due to having greater pod volume. Curve (d) has two distinct cycles of rise and fall, and a smaller maximum magnitude than curve (c). Accordingly, curve (d) corresponds to two pods traversing the slit scanning electrodes in series one after the other, where the two pods in series take twice as long to fully traverse the slit scanning electrodes than a single pod, but each pod has less capacitance than the total capacitance of two pods due to having smaller pod volume. In other words, aspect ratio of the waveform may reflect the number, size, and/or shape of pods measured. Similar to that described above, specific characterizations of shape and/or number of pods may be determined with a lookup table, a parametric model, a machine learning model, or any suitable method. Similarly, the controller may analyze the waveform to determine whether three, four, or more pods are traversing the slit scanning electrodes in any particular measurement region based on the slope and/or shape of the waveform. This determination may be useful, for example, to enable the controller to decide how to manipulate the pod or pods on each measurement region (e.g., apply a PDEP voltage as described below only if a single pod is present, do not apply a PDEP voltage as described below if multiple pods are present, etc.).

Furthermore, it should be understood that the slit scanning electrodes may additionally or alternatively be used to detect the absence of a pod in a measurement region (e.g., by measuring a waveform indicative of an open circuit, since the gap between the slit scanning electrodes would not be bridged by an absent pod).

### Interdigitated Electrodes

In some variations, at least one measurement region may include interdigitated electrodes, which may be configured to selectively retain or otherwise manipulate at least one pod in the measurement region by applying a holding force to the pod (e.g., based on the measured first characteristic of the pod) and/or measure a second characteristic of the pod, such as pod impedance (e.g., relating to the contents of the pod). Furthermore, the interdigitated electrodes of one measurement region may provide a separate measurement independent of other electrodes in other measurement regions. Accordingly, in some variations, the interdigitated electrodes may be referred to as a microelectrode “pixel” that provides a respective pod measurement value for processing.

As shown generally in the schematic of FIG. 8A, one variation of the interdigitated electrodes includes an active electrode 810 with a plurality of fingers and a ground electrode 820 with a plurality of fingers, where the fingers of the active electrode 810 alternate with the fingers of the ground electrode 820, with sufficient spacing between each finger such that the active and ground electrodes do not contact each other. The variation shown in FIG. 8 includes four fingers on each electrode. However, each electrode may include fewer (e.g., two, three) or more (e.g., five, six, or more) fingers. In some variations, the interdigitated electrodes may cover a region of about 500 μm by about 500 μm, though this may be varied in any suitable manner.

The interdigitated electrodes in a measurement region may be configured to retain at least one pod in the measurement region, so as to selectively hold the pod in place (e.g., for measurement and/or sorting purposes). In some variations, the electrodes 810 and 820 may be configured to generate a positive dielectrophoretic (PDEP) force. For example, an electrical voltage may be applied to the interdigitated electrodes to create an electric field between the active electrode 810 and the ground electrode 820. The electric field causes a PDEP attractive force to act upon the pod, thereby holding the pod in place in contact with the electrodes 810 and 820. As shown in the schematic of FIG. 8B, PDEP forces F may pull the pods against the electrodes 810 and 820, which may thereby cause the pods to deform and have increased surface area of contact with the electrodes. As described above, such a flattened shape may improve the quality of any measurements performed by the interdigitated electrodes.

Furthermore, it should be understood that forms of “activation” of the pods, other than pod retention, may be performed by the electrodes in the measurement regions. For example, assuming that a threshold PDEP voltage may be required to substantially immobilize a pod, a PDEP voltage lower than the threshold PDEP voltage may be applied to the electrodes of a measurement region in order to retard a pod's movement (e.g., cause the pod to decelerate, but not become stationary). As another example, a PDEP voltage significantly higher than the threshold PDEP voltage may be applied to the electrodes of a measurement region in order to accelerate a nearby pod.

As shown in FIG. 9, the controller 910 may govern operation of the interdigitated electrodes to retain at least one pod in a measurement region. Generally, the controller 910 may control application of a PDEP-causing voltage to the interdigitated electrodes, such as by controlling one or more switches connected to a PDEP voltage source 950 (or alternatively intermittently applying a PDEP voltage across a fixed connection to the interdigitated electrodes). For example, switches 930 and 934 connect the active electrode of respective interdigitated electrodes to the PDEP voltage source 950. The PDEP voltage source 950 may further be selectively operable with a switch 940 that connects the PDEP voltage source 950 to the rest of the control circuitry shown in FIG. 9. In some variations, the PDEP voltage may be about 3 V peak-to-peak applied at a frequency of about 50 Hz, though the voltage may have any suitable amplitude and/or frequency. With reference to row 1 shown in FIG. 9, a PDEP voltage may be applied to the interdigitated electrodes by toggling the switch 930 closed. When the PDEP voltage is applied while a pod is over the interdigitated electrodes, the resulting PDEP force may cause the pod to be retained over the interdigitated electrodes and within the measurement region. A similar arrangement may be repeated respectively for all measurement regions (e.g., row 1 through row M). In some variations, pod sorting may be accomplished by selectively retaining some pods with such as PDEP force while allowing other pods to circulate freely within the chamber, as further described below.

In some variations, the interdigitated electrodes in a measurement region may be configured to retain at least one pod based at least in part on a first characteristic measured by the slit scanning electrodes. For example, a characteristic (e.g., size or shape) of a pod entering a measurement region may be measured by the slit scanning electrodes as described above (e.g., to ensure multiple pods are not entering the measurement region simultaneously or together). Whether the interdigitated electrodes of the same measurement region become activated and retain the pod may be determined at least in part on the first characteristic. For example, the controller 910 may determine information relating to the size, number, and/or shape of a pod based on received electrical measurements (e.g., capacitance or voltage) from the slit scanning electrodes, as described above, and retain (or not retain) the pod based on the determined size, number, and/or shape of the pod as described above. In one example, if the controller 910 determines that a single pod has entered the measurement region, then the controller may cause application of a PDEP voltage to the interdigitated electrodes in that measurement region to retain the pod. In another example, if the controller 910 determines that a pod of a certain type (e.g., a certain shape) has entered the measurement region, then the controller 910 may cause application of a PDEP voltage to the interdigitated electrodes in that measurement region to retain the pod.

Additionally or alternatively, the electrodes 810 and 820 of FIG. 8 may be configured to measure a characteristic of a pod (e.g., a retained pod), such as pod volume or pod impedance. As shown in FIG. 9, the controller 910 may govern operation of the interdigitated electrodes to measure at least one characteristic in a measurement region. Generally, similar to that described above for measurement via the slit scanning electrodes, the controller 910 may control delivery of a measurement current to the interdigitated electrodes, such as by controlling one or more switches connected to the measurement current source 940 (or alternatively intermittently driving the measurement current across a fixed connection). For example, with reference to row 1, a measurement current may be applied across the interdigitated electrodes by toggling the switch 930 closed. In some variations, for example, the current may be a DC current generally on the order of about 1 μA, but may be any suitable kind of current for applying to the electrodes. When the measurement current is applied while a pod is over the interdigitated electrodes, a corresponding voltage (or other suitable signal) may be measured and subsequently analyzed by the waveform processor 920. A similar arrangement may be repeated respectively for all measurement regions (e.g., row 1 through row M).

For example, FIGS. 8C and 8D illustrate how a measured voltage waveform (FIG. 8D) may be analyzed in response to an applied measurement current (FIG. 8C) to determine a pod characteristic. As shown in FIG. 8C, at time t1, a measurement current (e.g., an AC signal) may be applied to the interdigitated electrodes, resulting in a measurable step up from v0 to v1 in the voltage waveform shown in FIG. 8D. The measurable step corresponds to real impedance that is measurable due to a relatively large double layer capacitance approximating a short circuit (similar to that described above with reference to FIG. 6A). The value of this real impedance depends on the impedance of the pod (e.g., pod contents).

As shown in FIG. 8D, following time t1, the voltage signal may increase generally linearly. For example, between time t1 and a future time t2, the voltage signal may increase generally linearly from v1 to v2. A slope of this portion of the waveform following time t1 corresponds to the double layer capacitance of the pod's boundaries. The value of the slope of the voltage waveform (e.g., the difference between v1 and v2 over the time period between t1 and t2) depends on the magnitude of the double layer capacitance.

Accordingly, the waveform processor may associate the step in the waveform with measured, real electrical impedance that is coupled in series with the double layer capacitance at the pod's boundaries, where the measured impedance may be correlatable to pod impedance. Furthermore, the waveform processor may associate the slope of the measured waveform with the amount of double layer capacitance at the pod's boundaries, which may be correlatable to pod volume. Such correlations between the measured waveform and pod impedance or pod volume may be performed with a lookup table. Additionally or alternatively, a parametric model or other suitable kind of correlating may be used to determine pod impedance and/or pod volume from the measured waveform. In yet other variations, pod impedance, pod volume, and/or other chemical or biological information may be interpreted with a machine learning model that correlates the measured waveform with such information. Such a machine learning model may be trained, for example, using suitable machine learning algorithms applied to training data derived from computer vision techniques, as described in further detail below.

In some variations, one or more pod characteristics may be measured as the result of measuring the varying pod impedance as a function of varying levels of PDEP voltage. Generally, the strength of the holding force retaining the pods may be controlled by adjusting the PDEP voltage, as PDEP force may generally be proportional to the square of the PDEP voltage. However, the measured impedance response of a pod to a particular PDEP voltage may vary depending on the pod's contents, size, etc. Accordingly, a curve or plot of the measured impedance response vs. applied PDEP voltage may be used to characterize a pod. For example, a first pod impedance may be measured while applying a first PDEP voltage to the electrodes, and a second pod impedance may be measured while applying second PDE voltage (different from the first PDEP voltage) to the electrodes, and so on to generate any suitable number of measured data points. The measured data points may be collected for the measured pod and analyzed in any suitable manner. In one example, measured data points may be matched (e.g., via best-fit techniques) to at least one known curve associated with a particular pod type having known characteristics, in order to classify the measured pod as the particular pod type. In another example, the measured data points collectively may be matched to a particular pod type using a suitable machine learning classification algorithm.

FIGS. 8E and 8F illustrate exemplary voltage measurements performed over time by the device for two samples of different test fluids. Sample A (FIG. 8E) was a first test solution having a high electrical conductance (447 μS/cm) and low impedance compared to Sample B (FIG. 8F), which was a second test solution having a low electrical conductance (23 μS/cm) and high impedance. Equal fluidic volumes of Sample A and Sample B were deposited in first and second wells, respectively, of the device. The voltage waveform shown in FIG. 8E was obtained by delivering a square wave measurement current to a measurement region in the first well containing Sample A, and measuring the subsequent voltage response. Similarly, the voltage waveform shown in FIG. 8F was obtained by delivering the same square wave measurement current to a measurement region in the second well containing Sample B. In both FIGS. 8E and 8F, the slope of the voltage waveform corresponds to double layer capacitance at the interface between the sample and electrodes in the measurement regions. However, FIG. 8F depicts a Vstep (B) that is higher than the Vstep (A) depicted in FIG. 8E. The Vstep (B) in FIG. 7B corresponds to the relatively high impedance of Sample B compared to that of Sample A.

Accordingly, FIGS. 8E and 8F generally illustrate how impedance of a sample may be identified based on the voltage offset Vstep in the measurement voltage waveform. Comparing the voltage offset Vstep to a predetermined threshold may, for example, be useful in determining sample characteristics.

In some variations, the device may be used to identify one or more binary characteristics of a sample entity based on the Vstep measurement (and accordingly, measured impedance of the sample entity). For example, in one illustrative application, the device may be used to determine the presence of a cell or agglutinate contained in a pod deposited in a well, by comparing a measured Vstep to a predetermined threshold. A measured Vstep that is above the predetermined threshold may indicate that at least one cell is present in a particular sample (because presence of a cell contributes to higher impedance of the sample containing the cell), while a measured Vstep that is below the predetermined threshold may indicate that no cell is present in a particular sample.

Furthermore, in some variations, the device may be used to identify characteristics of a sample entity based on how the Vstep measurement (and accordingly, measured impedance of the sample entity) compares against multiple predetermined thresholds. For example, in another illustrative application, the device may be used to identify the number of cells contained in a sample deposited in a well. A measured Vstep may be compared against multiple, progressively increasing thresholds to determine how many cells are present in a particular sample (because more cells can collectively contribute to higher impedance of the sample in a scaled manner). Additionally, by taking measurements of a sample at multiple points in time and tracking how many cells are determined to be present at each point in time, the device may be used to track cell growth rate.

Using at least the principles described above, exemplary applications of the devices and methods may measure a wide range of suitable sample characteristics. For example, cell counting (e.g., counting circulating tumor cells, white blood cells, and other kinds of cells, such as in multivariate index assays) may be useful in oncology and other therapeutic areas. As another example, measuring cell growth over time in the presence of antibiotics or antifungal substances may provide a measure for antibiotic susceptibility or antifungal resistance, which may be useful in drug development, diagnostics, and/or research applications. As yet another example, measuring agglutination of antibodies and antigens of interest (e.g., for testing strep throat, influenza, rabies, etc.) may be useful in diagnostic or other applications. Expanded descriptions of some of these and other exemplary applications are described in further detail below.

### Other Electrode Measurement Variations

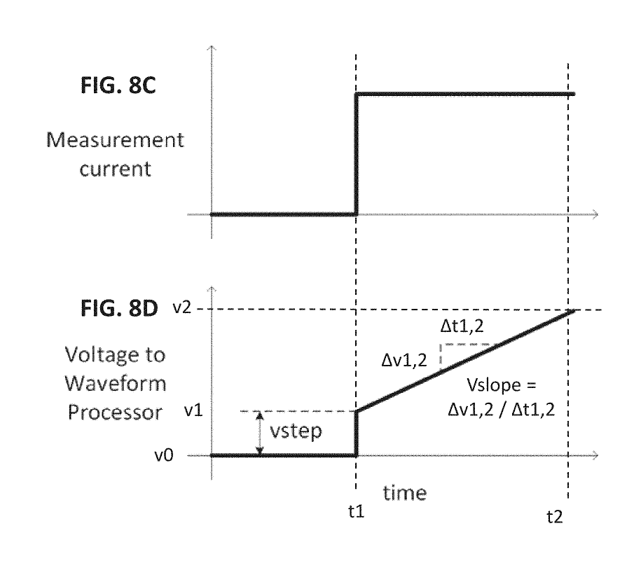

As described above, the measurement regions may be individually operable with switches are shown in FIG. 9. FIG. 10 illustrates another variation of an array 1000 of measurement regions that may be controlled with an addressing scheme. Array 1000 includes a matrix of n×m measurement regions. Each measurement region includes a respective set of interdigitated electrodes, and may include slit scanning electrodes as described above (though not shown in FIG. 10). Each measurement region may also include a respective transistor which allows for a “row, column” addressing scheme. For example, transistor 1010 is identified for the region in the 0^{th} row and 0^{th} column. Transistors may be built, for example, from amorphous silicon deposited onto a substrate using suitable thin film technology. In this example, in order to address a region and apply a voltage on the electrodes, digital-to-analog converters (DACs) DAC1-DACn may first set column voltages 1030 at source terminals for transistors in each of the DAC columns. Next, address drivers ADDR1-ADDRm may enable the gates 1040 of the transistors of a desired row in order to pass the DAC voltages onto the selected row of measurement regions. In some variations, the DACs may temporally cycle through PDEP voltages corresponding to the desired voltages to be applied to given rows of measurement regions. Address drivers sequentially or non-sequentially may enable the rows of measurement regions, thereby updating them with desired DAC voltages. The measurement regions may include a capacitor to retain the DAC voltages between updates.

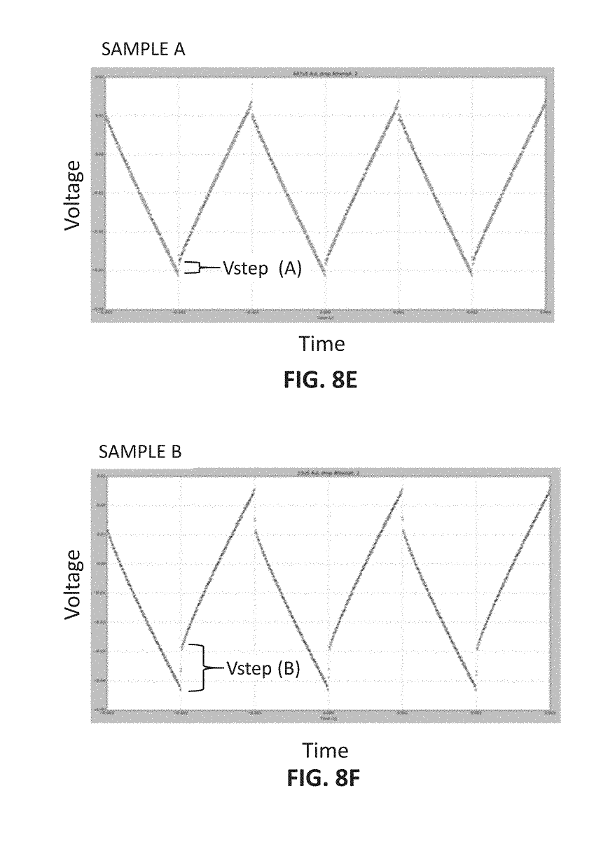

In yet other variations, other electrode arrangements in measurement regions may be included in the chamber of the device. For example, as shown in FIG. 11, instead of having interdigitated electrodes, an array 1100 of measurement regions may include ground electrodes 1110 incorporated in one or more planar sheets of electrically conductive material positioned adjacent (e.g., in front of, or behind, in the perspective shown in FIG. 11) to the fingers of active electrodes 1112. Small slits may be cut into the planar sheets, so as to allow for selective formation of electric fields between the active electrodes 1112 as the ground electrodes. The electrical field produced between the active electrodes 1112 and the ground electrodes may be a fringe field for producing a PDEP force in one or more selected measurement regions.

### Camera-Based Measurements

In some variations, as shown in FIG. 3, the assay device may include one or more cameras (e.g., shown schematically as camera 350), or other suitable image sensor configured to provide camera-based measurements of pods within the chamber 310. For example, at least a portion of one or more surfaces (e.g., upper surface, lower surface) of the chamber 310 may include a substantially optically transparent material through which a camera may view pods within the chamber. An entire surface may include an optically transparent material, or a surface may include “windows” or portions that include an optically transparent material. Suitable optically transparent materials include, for example, polycarbonate or glass. The material may, in some variations, include doped glass or patterned glass. For example, patterned glass may include patterned polymer thin films (e.g., with thickness ranging between about 5 μm and about 100 μm) such as polyimide. In some variations, at least one illumination source 360 (e.g., LEDs) may be arranged on a side of the chamber opposing the camera 350, so as to backlight the pods, and enhance contrast and overall visibility of pod contents. The illumination source 360 may, for example, provide diffuse lighting against the chamber, or a concentrated illumination beam for a specific region.

As shown in FIG. 3, one or more cameras may be mounted in an overhead location to provide a field of view including the chamber 310. It should be understood that in other variations, any suitable number of cameras may be mounted in any suitable orientation or position, including angled (e.g., in a corner of the chamber), along a sidewall, or along a lower surface of the chamber. Furthermore, as illustrated by the different camera positions shown in FIGS. 4A and 4B, the one or more cameras may be adjustable in position (e.g., X-direction, Y-direction, and/or Z-direction or depth), and/or in orientation. For example, a camera may be mounted on a track such that its position and/or orientation may be controlled by an actuated leadscrew or other suitable mechanism.

The camera may include an optical, thermal, and/or other suitable imaging sensor for capturing still images and/or video of pods that are in the chamber 310, where the still images and/or videos may be used for analysis of the pods. As described in further detail below, the still images and/or videos may be used to measure one or more characteristics of the pods, such as size, shape, chemical and/or biological information relating to content of the pods (e.g., color change in a reaction), and/or any suitable information. The camera images may be used, for example, in addition to or as an alternative to electrode measurements to measure one or more characteristics of pods. In particular, in some variations as shown in FIG. 3, the assay device may be configured to provide camera-based measurements using a camera 350, as well as electrode measurements using measurement regions 370 with electrodes (e.g., similar to those described above).

In an exemplary variation, the chamber may include a first surface (e.g., lower surface) including a flexible circuit board with measurement regions 370 having electrodes, and a second surface (e.g., upper surface) comprising an optically transparent material that is adjacent and spaced apart from the first surface. Pods passing between the first and second surfaces may thus be subject to both electrode measurements and camera-based measurements. Alternatively, the assay device may be configured to provide only electrode measurements using measurements regions 370, or only camera-based measurements using a camera 350. Furthermore, in some variations, as described below, camera images may be used to provide data for training and/or testing a machine learning algorithm that correlates electrode measurements to specific pod characteristics. Additionally, due to being compressed (e.g., into a “pancake”-like shape), a pod may be shaped to such that its contents are restricted to an approximate two-dimensional plane. As shown in FIG. 4D, this two-dimensional plane may be substantially coincident with the focal plane 430 of one or more cameras (e.g., cameras 412A and 412B), thereby improving detection of the pod contents (e.g., analytes) by the camera and improving the quality of camera-based measurements.

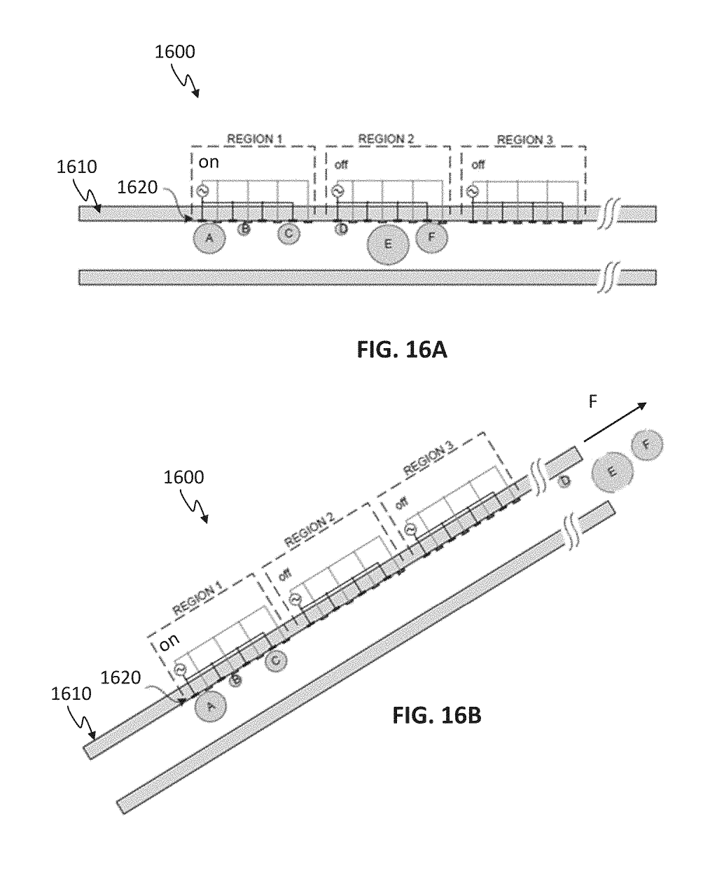

Various camera-based measurements may be performed using suitable computer vision techniques. For example, computer vision techniques may be used to measure size and/or shape of one or more imaged pods in the chamber. One exemplary illustration of such computer vision techniques is shown in FIG. 19. Following image processing such as background removal, contrast enhancement, etc., the boundaries of a pod P may be identified in a camera image using edge detection techniques or another suitable computer vision algorithm, and the identified boundaries may be identified on the camera image with a circle 1910 or other marking indicating the detected pod boundaries. Size of the pod P may be determined by measuring a diameter, circumference or other suitable dimension of the identified pod boundaries (e.g., diameter or circumference of the circle 1910), such as based on number of pixels, comparison to templates, etc. Similarly, this process may be performed on multiple pods such that overall pod polydispersity may be measured. In some variations, polydispersity may be measured substantially in real-time as the pods are introduced into the assay device. Pod location may furthermore be tracked substantially in real-time using similar computer vision techniques.

As another example, computer vision techniques may be used to detect and measure agglutination within a pod, thereby enabling analyte measurement in a variety of applications (e.g., drug discovery, research, diagnostic, etc.). For example, pods may include reagent particles (e.g., antibody-coated beads) specific to a target analyte that may or may not be present in a particular pod. If the analyte is not present in a pod, agglutination (clumping) between the analyte and the reagent particles will not occur. In contrast, if the analyte is present in a pod, such agglutination will occur.

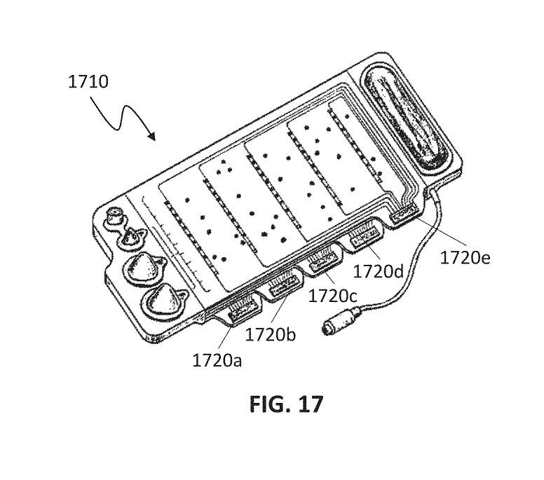

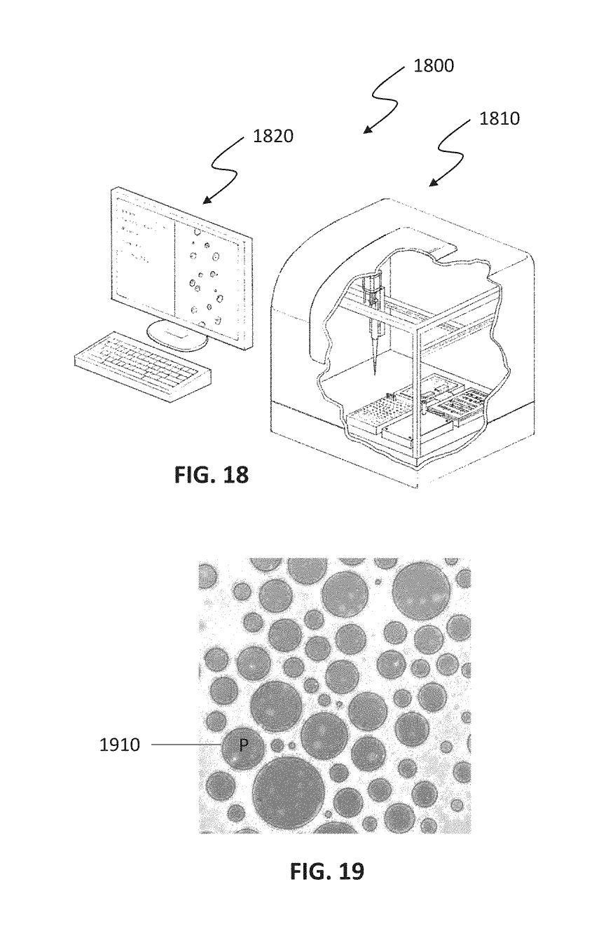

As described above, pods may be compressed within the assay device (e.g., between two surfaces) so as to be restricted into an approximate two-dimensional plane, and advantageously, this two-dimensional plane may be substantially coincident with the focal plane of one or more cameras. When viewed along an axis generally orthogonal to the focal plane, a pod may have a different optical appearance depending on whether agglutination is present. For example, as shown in the schematic of FIG. 20A, reagent particles may appear more diffuse or distributed in a pod with substantially no agglutination present. In contrast, substantial clumping of reagent particles will be apparent in a pod with agglutination present, and the agglutination will tend to result in fewer, larger clumps. For example, as shown in the schematic of FIG. 21A, the agglutination may tend to lead to the appearance of a single clump “A” (approximating a “one-dimensional dot”) within the two-dimensional focal plane.

In some variations, a computer vision technique for detecting and/or measuring agglutination in a pod may be based at least in part on distribution of pixel darkness or grayscale intensity in an optical image of the pod. For example, as shown in FIG. 20B corresponding to a non-agglutinated pod, a histogram of grayscale pixel darkness in an optical image of the pod may generally approximate a low, broad bell curve. This low, broad bell curve generally corresponds to the distributed reagent particles depicted in the optical image with pixels having a broad, lower range of individual grayscale darkness. In contrast, as shown in FIG. 21B corresponding to an agglutinated pod, a histogram of grayscale pixel darkness in an optical image of the pod may generally approximate a “sharp peaked” curve. This “sharp peaked” curve generally corresponds to the larger, clumped reagent particles collectively depicted in the optical image with pixels having a narrower, higher range of grayscale darkness. Thus, the shape of the pixel grayscale histogram for an image of a pod may be analyzed in order to determine whether agglutination is present in the pod. Additionally or alternatively, the mean pixel grayscale value for an image of the pod may be analyzed to determine whether agglutination is present in the pod. For example, if the mean pixel grayscale value is lower than a predetermined threshold, then the pod may be deemed as having no agglutination present. As another example, if the mean pixel grayscale value is higher than a predetermined threshold, then the pod may be deemed as having agglutination present.

Additionally or alternatively, a computer vision technique for detecting and/or measuring agglutination may be based at least in part on detected size of entities (reagent particles, agglutinated clumps) within an imaged pod. Suitable edge detection techniques (e.g., pixel intensity thresholding) may be applied to an image of a pod, to locate boundaries, and thus size, of entities within the imaged pod. As shown in FIG. 20C corresponding to a non-agglutinated pod, a histogram of size of entities in the pod may tend to indicate many smaller-sized entities. In contrast, as shown in FIG. 21C corresponding to an agglutinated pod, a histogram of size of entities in the pod may tend to indicate fewer, larger entities.

Similarly with either of the above techniques, a measurement of the degree or amount of agglutination may be performed. For example, detection of fewer, larger clumps (which may be indicated by more pixels having a darker grayscale intensity, for example) may be indicative of greater agglutination.

In yet other variations, computer vision techniques may be used to characterize dynamic qualities of pod contents over time. For example, a rate of agglutination may be measured by comparing sequential camera-based measurements of agglutination as described above. As another example, change in agglutinate size in response to a mechanical input (e.g., rate and/or degree of separation or breaking up of clumps), such as agitation of the assay device, may be a useful metric in characterizing a pod and its contents.

Accordingly, in some variations, an assay device may perform camera-based measurements of pod characteristics such as size or agglutination (in addition to or as an alternative to electrode measurements described above), using suitable computer vision techniques such as those described above.

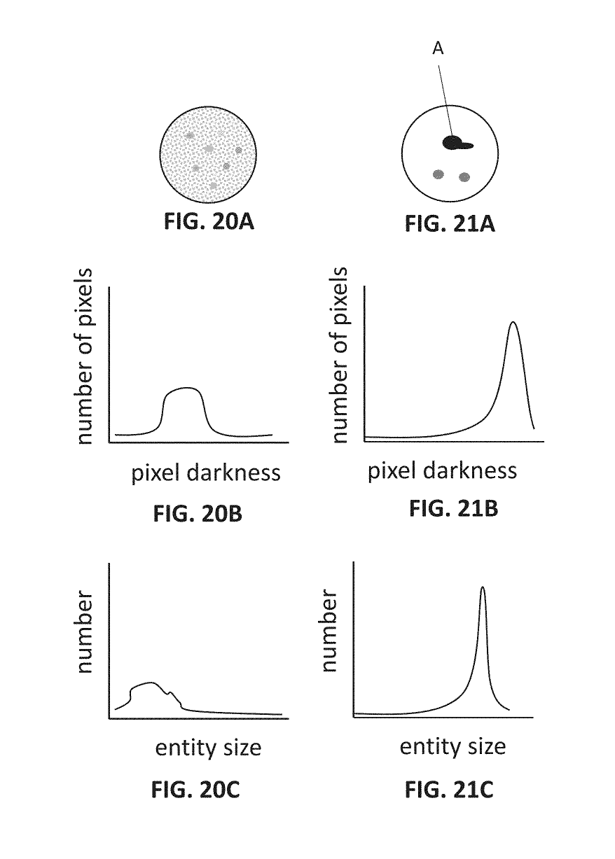

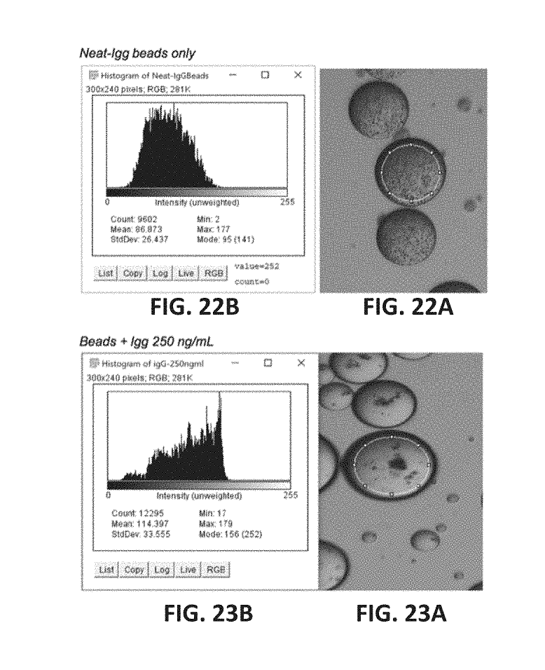

FIGS. 22-23 are exemplary images and data illustrating camera-based detection of non-agglutination and agglutination in pods. In particular, FIGS. 22A and 23A are camera images of pods including at least antibody-coated beads specific to immunoglobulin G (IgG). However, the pods in FIG. 22A have 0 ppm of IgG and thus exhibit no agglutination, while the pods in FIG. 23A have 250 ppm of IgG and thus do exhibit some degree of agglutination. FIG. 22B is a histogram of pixel grayscale intensity for the image of the encircled pod shown in FIG. 22A, and accordingly has a generally bell-shaped curve that may be interpreted to detect the non-agglutination of the pod. FIG. 23B is a similar histogram of pixel grayscale intensity for the image of the encircled pod shown in FIG. 23A, and accordingly has a generally “sharp peaked” curve (notably tending toward a higher mean pixel grayscale intensity compared to FIG. 22B) that may be interpreted to detect the agglutination of the pod.

### Methods for Processing Sample Entities

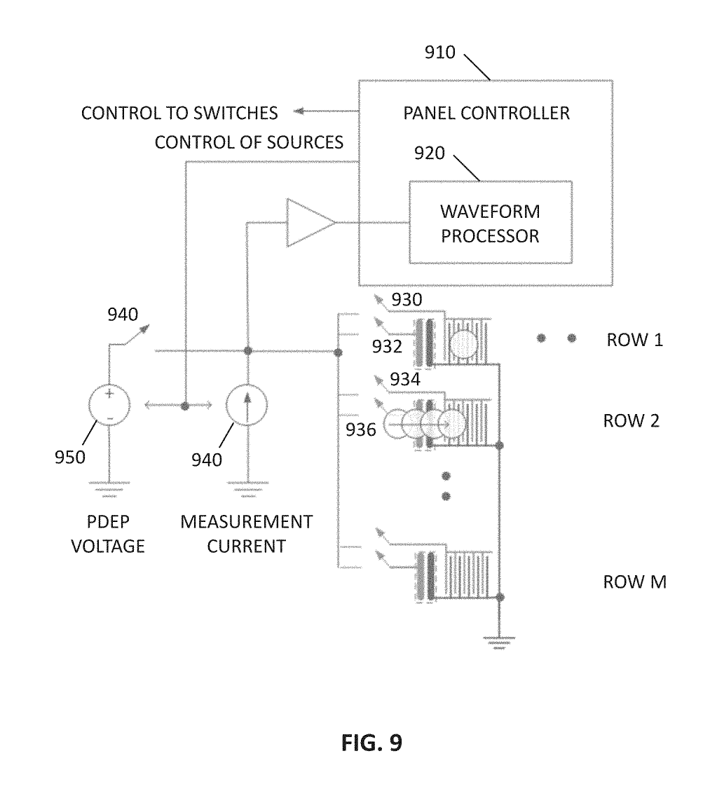

Generally, as shown in FIG. 12, a method 1200 for processing sample entities includes at least some of the steps of receiving a plurality of sample entities 1210 in a chamber comprising an array of measurement regions, measuring a first characteristic of at least one sample entity 1220 in a measurement region, and retaining the sample entity 1240 in the measurement region based at least in part on the measured first characteristic. In some variations, the method 1200 may include measuring a second characteristic of the retained sample entity 1250, and/or sorting the sample entity 1260. In some variations, the sample entities may be polydisperse. Furthermore, in some variations, the method may include tracking the sample entity with a virtual tag 1230 within the chamber, where the virtual tag may be associated with a particular sample entity and may store information relating to the sample entity such as identifying information or various one or more characteristics about the sample entity (e.g., size, shape, contents, etc.).

### Chamber Filling

A plurality of pods or other suitable sample entities may be received in a chamber (e.g., similar to those described above) as they are passed into the chamber by a fluidic pump system or in any suitable manner. In some variations, the pods may be transferred into the chamber until the chamber is substantially full (e.g., at least one pod is in contact with all or nearly all of the measurement regions in the chamber). One example of monitoring fill level is using the electrodes in the measurement regions (e.g., split scanning electrodes) to determine the presence or absence of pods on each measurement region at various locations within the chamber. Additionally or alternatively, fill level may be monitored by measuring volumetric flow rate within the fluidic pump system.

In some variations, flow rate may be gradually ramped up (e.g., to quickly fill the chamber and expedite processing of pods) at the beginning of pod transfer into the chamber. Additionally or alternatively, the flow rate of the pods into the chamber may be gradually ramped down near the end of the pod transfer (e.g., when the chamber is nearly filled, such as at about 90% capacity). With the reduced flow rate, the pods may tend to travel within the chamber at a slower speed suitable for performing electrode measurements and otherwise processing the pods. When the chamber is determined to be sufficiently full, the flow rate of the pods may be halted.

### Measurements and Activation

In some variations, measuring a first characteristic of a sample entity 1220 may include performing a measurement with slit scanning electrodes such as those described above. For example, the measurement may be performed by applying a measurement current to electrodes in a measurement region when at least one pod is traversing the measurement region (e.g., is in motion), and measuring a voltage waveform (or other electrical measurement) that may be correlated to a pod size and/or shape, as described in further detail above. In other variations, measuring a first characteristic of a sample entity 1220 may include performing a camera-based measurement such as those described above. For example, the measurement may be performed by utilizing computer vision techniques (e.g., based on edge detection, pixel grayscale intensity, etc.) to determine pod size and/or shape, as described in further detail above.

Retaining the sample entity 1240 in a measurement region may include applying a voltage to electrodes (e.g., interdigitated electrodes or other electrodes of suitable shape and pattern) in the measurement region which may cause a holding force (e.g., a PDEP force) to attract the pod with enough force to slow or substantially immobilize the pod on the measurement region. A pod may be retained in a measurement region based on the measured first characteristic, which may, for example, determine whether multiple pods are present on the measurement region and/or the size or shape of the pod. In some variations, only single pods of a certain type may be retained on the measurement region.

Since measurement regions may be operated independently of one another, pods may be selectively retained in any suitable temporal and/or spatial manner. For example, various pods may be retained by the electrodes in a desired spatial pattern of pods within the array of measurement regions. At least some various pods may be retained in the desired spatial pattern either serially (e.g., in sequence), and/or at least some various pods may be retained in the desired spatial pattern substantially simultaneously (e.g., in parallel).

Furthermore, it should be understood that forms of “activation” or manipulation of the pods other than pod retention may be performed by the electrodes in the measurement regions. For example, assuming that a threshold PDEP voltage may be required to substantially immobilize a pod, a PDEP voltage lower than the threshold PDEP voltage may be applied to the electrodes of a measurement region in order to retard a pod's movement (e.g., cause the pod to decelerate, but not become stagnant). As another example, a PDEP voltage significantly higher than the threshold PDEP voltage may be applied to the electrodes of a measurement region in order to accelerate a nearby pod.

Additional pod characteristics may be measured, such as when a pod is retained on (or otherwise activated by) a measurement region. For example, second, third, or additional measurements may be performed on the retained pod by applying a measurement current and analyzing the resulting waveform that is indicative of pod impedance and reflect chemical and/or biological information about the pod contents. Additionally or alternatively, the method may include measuring one or more pod characteristics with at least one image sensor (e.g., camera).

In some variations, the method may include performing a first measurement (e.g., impedance measurement), performing a second measurement after a predetermined period of time, and comparing the first and second measurements in order to determine a pod characteristic.

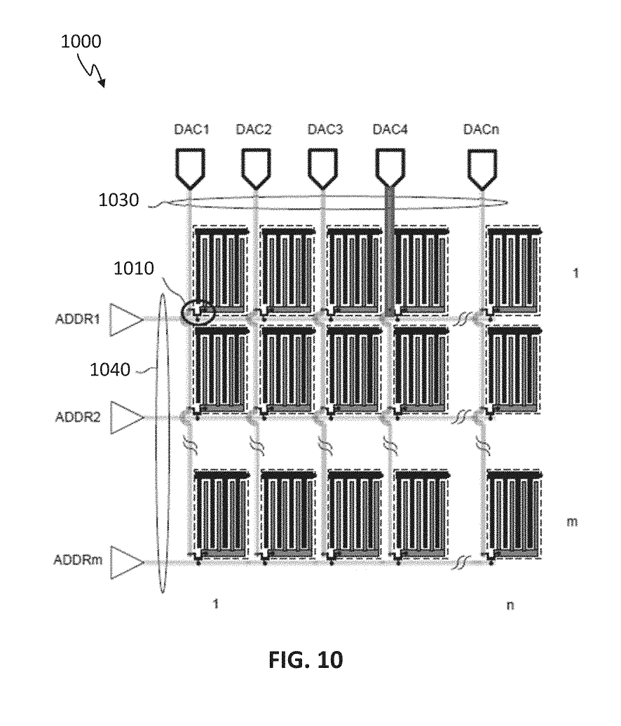

Furthermore, additional pod characteristics may be measured even when a pod is not retained or otherwise “activated” or manipulated as described above. For example, a differential in pod impedance between when a pod is not retained and when the pod is retained may, in itself, serve as a pod characteristic. As shown in the schematic of FIGS. 13A and 13B, for example, an earlier measurement may indicate pod characteristics when the pods are not retained on measurement regions as shown in FIG. 13A (e.g., no PDEP voltages applied), while a later measurement may indicate pod characteristics when the pods are retained as shown in FIG. 13B. A differential between the earlier and later measurements may be a notable pod characteristic. Between FIGS. 13A and 13B, the contents of pod P1 demonstrate a relatively high packing density affinity, in that the pod contents tend to pack more densely in response to the PDEP force. In contrast, the contents of pod P2 demonstrate a relatively low packing density affinity, in that the pod contents tend to not alter in packing density in response to the PDEP force. Accordingly, packing density affinity as reflected in the differential measurements may be a notable pod characteristic. As another example, between FIGS. 13A and 13B, the size (diameter) of pod P3 increases, and the size differential may be a notable pod characteristic.

Additionally or alternatively, in some variations, the retention of the pods may be omitted. For example, in variations in which camera-based measurements of pod characteristics are performed (e.g., similar to those techniques described herein), such camera-based measurements may be sufficient on their own to provide insight into their content (e.g., agglutination) and/or combined with electrode-based measurements described herein.

### Tracking

In some variations, the method may include tracking at least one sample entity. Tracking the sample entity 1230 may include tracking location and/or trajectory of the sample entity within the chamber. For example, once a pod is identified as present on a particular measurement region with slit scanning electrodes or other measurement electrodes, a controller (e.g., controller 910 described above with respect to FIG. 9) may create a virtual tag that is associated with the pod. As another example, a virtual tag may be created once a pod is identified in an image. In some variations, the virtual tag may include a vector or other data construct stored in any suitable memory device, and may include relevant identifying information about the pod (e.g., size, shape, other identifying characteristics that may be unique to the pod, etc.). Information about the pod may be continually gathered by measurement regions and/or cameras as the pod circulates within the chamber and interacts with various measurement regions. For example, after measuring a pod in a first measurement region and storing at least one identifying characteristic about a pod in a virtual tag, the pod may be subsequently measured and recognized in a second measurement region based on the identifying characteristic. As another example, after performing a camera-based measurement of a pod and storing at least one identifying characteristic about a pod in a virtual tag, the pod may be subsequently measured and recognized in a second camera image based on the identifying characteristic. Any additional measurements in the second measurement region (or in third, fourth, and any subsequent measurement regions interacting with the pod at the pod circulates within the chamber) and/or measurements from an image sensor may be further added to the virtual tag, along with a timestamp indicating time of measurement (or an index indicating numerical order of measurement, etc.). Unlike sample entity labels in conventional assays that are consumed through redox or other chemical reactions, a virtual tag associated with a pod may be stored and recalled an unlimited number of times, such as for purposes of tracking pods or identifying pods disposition during sorting as described below.

### Sorting

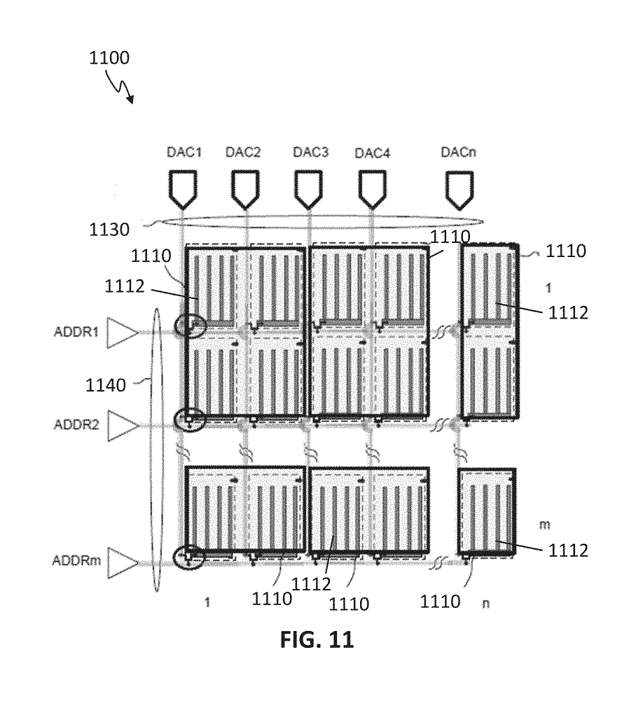

In some variations, the method may include sorting at least one sample entity. Sorting pods may include selectively retaining a first portion of the pods in the chamber with a holding force (e.g., PDEP force) and allowing a second portion of the pods to exit via one or more outlets of the chamber. For example, as generally shown in the schematic of FIG. 14A, a chamber may include a plurality of pods. Although the pods are depicted as substantially monodisperse, it should be understood that in other variations, the pods may be polydisperse. Pods in rows R1 and R4 may be desired to be isolated from pods in rows R2 and R3. Accordingly, pods in rows R1 and R4 may be retained by application of a PDEP voltage on underlying electrodes/measurement regions (activated as shown in FIG. 14B), while pods in rows R2 and R3 may remain free. As shown in FIG. 14C, the free pods in rows R2 and R3 may be removed from the chamber (e.g., by fluidic currents and/or tilting of the chamber to leverage gravity and buoyancy effects) via one or more outlets, such that only pods in rows R1 and R4 remain. Generally, sorting may be based on measured pod content, pod size, pod shape, pod location within the chamber, and/or any suitable characteristic.

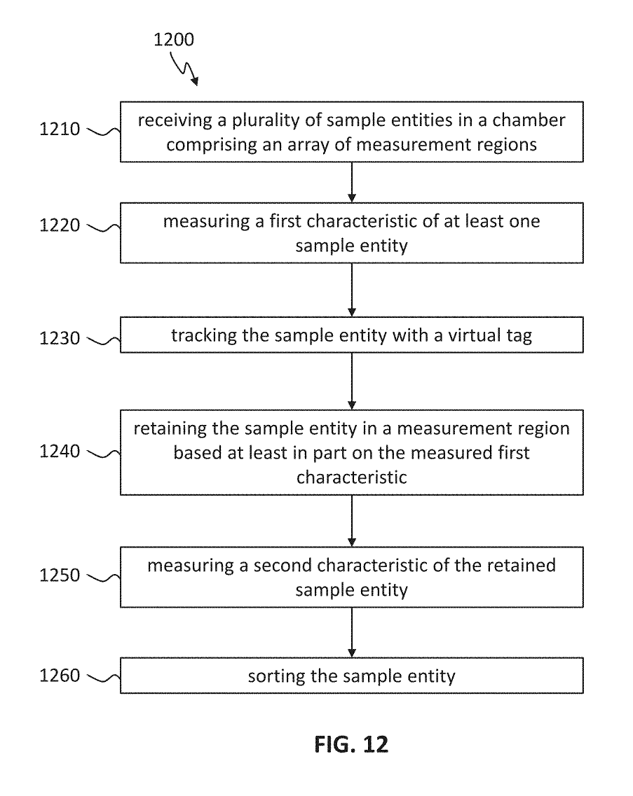

During sorting, retained or otherwise activated pods may be in any desirable spatial pattern, as each measurement region may independent be controlled to retain or not retain a pod. For example, as shown in FIG. 15A, pods located generally on an upper side and a lower side of the chamber may be retained, while free pods generally located along a central region of the chamber may be allowed to exit the chamber. As another example, as shown in FIG. 15B, selected pods to be retained may be arranged in a manner so as to allow free pods to follow a generally serpentine path out of the chamber. It should be understood that these retention patterns are merely exemplary, and any suitable pod activation pattern may be used to sort the pods.

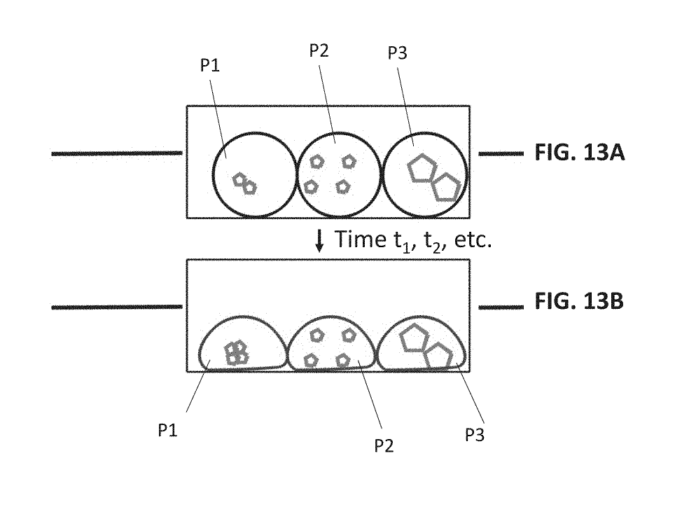

Free pods may be manipulated to exit the chamber in one or more various manners. In some variations, gravity and buoyancy effects may be used at least in part to direct pods toward one or more outlets of the chamber. For example, in some variations the pods may be less dense than their surrounding medium, such that the pods tend to float or rise up within the medium. As shown in the schematic of FIG. 16A, pods A-F (represented graphically here as polydisperse spheres) are located within a chamber 1610 having an array of measurement regions with electrodes 1620. Pods A-C are retained on measurement region 1, such as by virtue of a PDEP force, while pods D-F are positioned on measurement region 2 but are free to circulate. In FIG. 16B, the chamber 1610 is tilted. Because pods D-F are less dense than their surrounding medium and are not held in place on measurement region 2, pods D-F are directed upwards out of the chamber by buoyancy force F. In contrast, pods A-C are held in place on measurement region 1 and are prevented from exiting the chamber, thereby sorting pods D-F from pods A-C. In some variations, the chamber may be tilted in multiple directions to direct pods to exit from different outlets of the chamber. For example, different sets of pods may be released in stages, where in each stage the chamber tilts in a different direction so the free pods exit through a different outlet.

In some variations, sorting may additionally or alternatively include introducing flow currents (e.g., via pressure sources such as one or more fluidic pumps, pipetting action, and/or other suitable pressure source) into the chamber to flush free pods from the chamber. The flow currents may be positioned at various suitable locations to direct pods toward particular outlets. In some variations, different sets of pods may be released in stages, where in each stage a different flow current urges the released pods toward a different outlet

It should be understood that tilting and flow currents may be used in series and/or in parallel in order to sort pods in any suitable manner. Additionally or alternatively, chamber walls, surface etchings, partitions, and/or any suitable structural features of the chambers may be used to redirect and sort pods.

### Training an Electrode Measurement Model

As described above, in some variations, electrode measurements may be correlated to pod characteristics through an electrode measurement model trained using a suitable machine learning algorithm. For example, the electrode measurement model may be trained using suitable supervised or unsupervised machine learning algorithm such as a neural network algorithm, decision trees, vector machines, etc. In some variations, training data (e.g., feature vectors) for the electrode measurement may include known characteristics and empirical electrode measurement data relating to the same set of one or more pods. The pod characteristics forming part of the training data may be determined, for example, through computer vision techniques, as described below. By applying a machine learning algorithm to the training data, relationships between the electrode measurements and pod characteristics may be developed and embodied in the electrode measurement model. Furthermore, the trained electrode measurement model may be tested and iterated upon by using test data of the same type as the training data (e.g., known characteristics and electrode measurement data of a test set of pods).

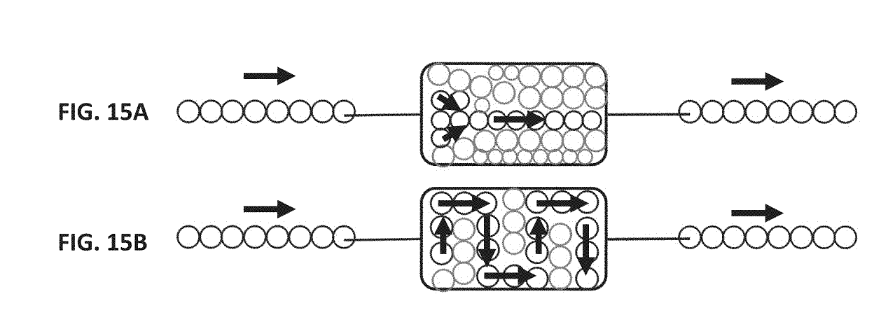

FIG. 18 is a schematic illustration of an assay development system 1800 including an assay device 1810 having an array of measurement regions with electrodes similar to that described above, and a control system 1820 (e.g., including one or more processors) for operating the assay device 1810. The assay device 1810 may further include one or more cameras (e.g., providing optical images, thermal images, etc.) directed toward the measurement regions such that pods introduced into the assay device 1810 may be sensed and/or measured with cameras and electrodes as described above. The assay device 1810 may include an electromechanical arrangement for introducing the pods into the assay device, such as for developing training data.

In some variations, training data may be developed at least in part by measuring an electrical characteristic (e.g., pod impedance) of at least one pod introduced into the assay device 1810, receiving one or more images of the pod, and measuring at least one characteristic of the pod by analyzing the one or more images with computer vision techniques. In other words, as pods move through the assay device 1810, the pods may be measured with both electrodes and cameras substantially in real-time. For example, suitable computer vision techniques (e.g., edge detection techniques) may be used to determine pod size, calculate polydispersity or size variance within a set of pods, and interpret biological information relating to the pod (e.g., pod contents).

Furthermore, it should be understood that in some variations, the assay development system 1800 shown in FIG. 18 may additionally or alternatively be used to process pods for research and/or diagnostic purposes, not just for development of training data. For example, both electrode measurements and camera measurements of pods may be obtained to provide validation and/or redundancy in determining characteristics of the pods.

### Exemplary Applications

In one exemplary application of the devices and methods described herein, pods including yeast cells may be introduced into the chamber. The electrical impedance of the pods (and of the yeast cells contained therein) may be measured to provide a first (e.g., baseline) impedance measurement. The electrical impedance of the pods may be subsequently measured a second time after a predetermined period of time to provide a second impedance measurement. As growth of the yeast cells affects impedance, the difference between the first and second impedance measurements may be used to determine growth of the yeast culture within each pod individually. Additional impedance measurements may be taken to further establish trends in culture growth in each pod. Furthermore, statistical data generated from information about all of the pods may be used to determine the yeast growth trajectory.

In another exemplary application of the devices and methods described herein, pods including bacteria cells may be introduced into the chamber. Varying concentrations of a water-soluble antibiotic may be dissolved in the aqueous content of each pod. For example, each of a first set of pods may include a first concentration of soluble antibiotic and a bacterium, each of a second set of pods may include a second concentration of soluble antibiotic and a bacterium, and so on. The electrical impedance of the pods may be measured to provide a first (e.g., baseline) impedance measurement. The electrical impedance of the pods may be subsequently measured a second time to provide a second impedance measurement. As cell division (e.g., resulting number of cells) affects impedance of the pods, the difference between the first and second impedance measurements may be used to assess the bacteria's varied response to different concentrations of antibiotic. Additional impedance measurements may be taken to further establish trends in cell division and the bacteria's extended response to the antibiotic. Accordingly, the assay device may, for example, be used to characterize the bacteria's resistance to the antibiotic and/or select an effective antibiotic concentration for therapeutic use.

In another exemplary application of the devices and methods described herein, pods including cells from different cell lines may be introduced into the chamber. The different cell lines may, for example, be genetically altered (e.g., to express variants of different proteins on their surfaces). A suitable drug or other small molecule (whose interaction with cells is being investigated) may be contained within each pod along with a cell. For example, each of a first set of pods may include a drug and a cell having a first genetic alteration, each of a second set of pods may include the drug and a cell having a second genetic alternation, and so on. The electrical impedance of the pods may be measured to provide a first (e.g., baseline) impedance measurement. The electrical impedance of the pods may be subsequently measured a second time to provide a second impedance measurement. As response of the cells (e.g., through morphology, agglutination, etc. as the result of absorbing the drug) may affect impedance of the pods, the difference between the first and second impedance measurements may be used to assess how the different kinds of cells respond to the presence of the drug. Additional impedance measurements may be taken to further establish trends in the cells' reactions. Accordingly, the assay device may, for example, be used to characterize the effects of each genetic alteration among the cell lines with respect to drug response, and these effects may serve as models for pharmaceutical, human, plant, microbiological, etc. applications.

In another exemplary application of the devices and methods described herein, pods including antibody-coated latex beads may be introduced into the chamber. The beads may be polydisperse. The antibodies may correspond to a specific antigen of interest (e.g., an antigen for testing for strep throat or influenza, a prostate-specific antigen for testing for prostate cancer or other protein, etc.). A patient sample may be introduced into each pod for testing of the presence of the antigen of interest. The electrical impedance of the pods may be measured to provide a first (e.g., baseline) impedance measurement. In some variations, the pods may be agitated to encourage mixing of contents within the pod. The electrical impedance of the pods may subsequently be measured a second time to provide a second impedance measurement. As agglutination resulting from binding of the antibodies and any antigens may affect the impedance of the pods, the difference between the first and second impedance measurements may be used to assess agglutination or self-aggregation of the pods. Accordingly, the assay device may, for example, be used to test for the presence of the antigen of interest, and thereby diagnose the patient for the associated condition (e.g., strep throat, influenza, prostate cancer, etc.). In other variations, the difference between the first and second impedance measurements (and any additional impedance measurements, and/or the time elapsed between the impedance measurements) may be used to assess colloidal stability resulting from the surface binding properties of the agglutinated pods.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for processing sample entities, comprising:
a chamber comprising a surface having an array of measurement regions, wherein at least one measurement region comprises a first set of one or more electrodes and a second set of one or more electrodes,
wherein the first set of electrodes is configured to measure a first characteristic of a sample entity when the sample entity is traversing the first set of electrodes, and
wherein the second set of electrodes is configured to selectively retain the sample entity in the at least one measurement region based at least in part on the measured first characteristic.

2. The system of claim 1, wherein the first characteristic is measured based at least in part on a measured double layer capacitance of the sample entity.

3. The system of claim 2, wherein the first characteristic comprises at least one of size and shape of the sample entity.

4. The system of claim 1, wherein at least one of the first set of electrodes is larger than a diameter of the sample entity.

5. The system of claim 1, wherein the first set of electrodes comprises at least two elongated electrodes separated by a scanning distance.

6. The system of claim 1, wherein at least one of the second set of electrodes is configured to retain the sample entity with a dielectrophoretic force.

7. The system of claim 1, wherein the second set of electrodes comprises interdigitated electrodes.

8. The system of claim 1, wherein at least one of the first set of electrodes and the second set of electrodes is configured to measure a second characteristic of the sample entity based at least in part on a measured electrical impedance of the sample entity coupled with a double layer capacitance of the sample entity.

9. The system of claim 1, further comprising an image sensor configured to measure a second characteristic of the sample entity.

10. The system of claim 1, wherein the array of measurement regions comprises a two-dimensional grid.

11. The system of claim 1, wherein the system is configured to process polydisperse sample entities.

12. The system of claim 1, wherein the chamber comprises a first surface and a second surface offset from the first surface by a gap distance configured to compress a sample entity between the first and second surfaces into a pod.

13. A system for processing at least one sample entity, comprising:
a chamber comprising an array of measurement regions, wherein at least one measurement region comprises at least one electrode larger than a diameter of the sample entity; and
wherein the at least one electrode is configured to measure a characteristic of the sample entity when the sample entity is traversing the at least one electrode.

14-18. (canceled)

19. A method for processing sample entities, comprising:
receiving a plurality of sample entities in a chamber comprising an array of measurement regions, wherein at least one measurement region comprises a plurality of electrodes;
measuring a first characteristic of at least one sample entity with at least a portion of the electrodes as the sample entity traverses the portion of the electrodes; and
retaining the sample entity in the at least one measurement region based at least in part on the measured first characteristic.

20. The method of claim 19, wherein receiving the plurality of sample entities comprises deforming at least one sample entity to increase the area of contact between the sample entity and a surface of the chamber.

21. The method of claim 19, wherein measuring the first characteristic comprises delivering an alternating current from the portion of the electrodes to the sample entity.

22. The method of claim 21, wherein measuring the first characteristic comprises periodically delivering the current as the sample entity traverses the portion of the electrodes.

23. The method of claim 19, wherein retaining the sample entity in the at least one measurement region comprises generating a dielectrophoretic force with at least a portion of the electrodes.

24. The method of claim 19, further comprising measuring a second characteristic of the retained sample entity.

25. The method of claim 24, wherein the second characteristic relates to at least one of number, size, morphology, and division of one or more cells within the sample entity.

26. The method of claim 24, wherein the second characteristic relates to degree of agglutination within the sample entity.

27. The method of claim 24, wherein measuring the second characteristic comprises delivering an alternating current to the sample entity and measuring an impedance coupled with a double layer capacitance of the sample entity.

28. The method of claim 19, further comprising creating a virtual tag associated with the sample entity, wherein the virtual tag comprises at least the first characteristic of the sample entity.

29. The method of claim 19, further comprising sorting the plurality of sample entities.

30. The method of claim 29, wherein sorting comprises selectively retaining a first portion of the sample entities on one or more measurement regions.

31. The method of claim 30, wherein sorting comprises introducing a fluidic current into the chamber to manipulate a second portion of the sample entities different from the first portion of the sample entities.

32. The method of claim 19, wherein the plurality of sample entities are polydisperse.

33. The method of claim 19, further comprising compressing at least one of the sample entities into a pod in the chamber.

34. A system for processing sample entities, comprising:
a chamber comprising a first surface and a second surface offset from the first surface, wherein the first and second surfaces are configured to compress a sample entity into a flattened pod,
wherein at least one of the first and second surfaces comprises an optically transparent material.

35-48. (canceled)
